(19) United States
(12) Patent Application Publication
(10) Pub. No.: US 2003/0180766 A1
(43) Pub. Date: Sep. 25, 2003

(54) Method, system and knowledge repository for identifying a secondary metabolite from a microorganism

(75) Inventors: Chris M. Farnet, Outremont (CA); Alfredo Staffa, Saint-Laurent (CA); Brian O. Bachmann, Westmount (CA); James B. McAlpine, Westmount (CA); Emmanuel Zazopoulos, Montreal (CA); Zhizi Zhao, Pierrefonds (CA); Sai Man Wong, Saint-Laurent (CA); Nicolas Desjardins, Pointe-Claire (CA)

Correspondence Address: TIMOTHY BUTTS, 1128 W. 76TH TER APT #6, SHAWNEE, KS, 66214, US

(73) Assignee: Ecopia BioSciences, Inc.

(21) Appl. No.: 10/350,341

(22) Filed: Jan. 24, 2003

Related U.S. Application Data
(60) Provisional application No. 60/350,369, filed on Jan. 24, 2002.
(60) Provisional application No. 60/398,795, filed on Jul. 29, 2002.
(60) Provisional application No. 60/412,580, filed on Sep. 23, 2002.

(51) Int. Cl. C12Q 1/68; G01N 33/554; G01N 33/569; G06F 19/00; G01N 33/48; G01N 33/50

(52) U.S. Cl. 435/6; 435/7.32; 702/19

(57) ABSTRACT

The invention relates to a method and system for identifying a secondary metabolite synthesized by a target gene cluster within a microorganism. A putative or confirmed function is attributed to a gene within the gene cluster, and an extract from the microorganism is obtained which is suspected to contain the secondary metabolite synthesized by the gene cluster. The extract is then assessed for chemical, physical or biological properties, and the metabolite is identified and optionally isolated. Further, the invention provides a knowledge repository in which gene cluster information is linked to secondary metabolite production data. The invention further relates to a graphical user interface for accessing the knowledge repository, and a memory for storing data, having a data structure that is stored in the memory.

### RELATED APPLICATIONS

This application claims the benefit of U.S. Provisional Application No. 60/350,369 filed on Jan. 24, 2002; U.S. Provisional Application No. 60/398,795 filed on Jul. 29, 2002; and U.S. Provisional Application No. 60/412,580 filed on Sep. 23, 2002. The teachings of the above applications are incorporated herein by reference in their entirety.

### FIELD OF THE INVENTION

The present invention relates generally to a bioinformatics method and system for identifying products of secondary metabolism in a microorganism.

### BACKGROUND OF THE INVENTION

Natural product metabolites are widely used as bioactive compounds, dyes, plasticizers, surfactants, scents, flavorings, drugs, herbicides, pesticides and lead compounds for such applications. Improvements in methods of discovery of natural product metabolites would be of benefit to many fields. One field of natural products in which there is an urgent need for improved discovery methods is natural product drug development. While the rate of discovery of new antibiotics has dropped significantly over the past few decades, analysis of antibiotic discovery rates suggests that a large number of antibiotics remain to be discovered from actinomycete natural product metabolites (Watve et al., (2001) Arch. Microbiology 176:386-390). Recent genome sequencing studies demonstrate that the ability of actinomycetes to produce bioactive secondary metabolites has been vastly underestimated. For example, 25 secondary metabolite gene clusters were identified in the genome of Streptomyces avermitilis by whole genome shotgun sequencing of S. avermitilis despite the fact that the organism had previously been reported to produce only two natural products (Omura et al. Proc. Natl. Acad. Sci. USA, 98, 12215-12220). Likewise a genome project of Streptomyces coelicolor demonstrated that the S. coelicolor genome contains biosynthetic gene clusters for 12 or more natural products while the organism was previously known to product three or four natural products (Bentley, S. D. et al., Nature, 147, 141-147 (2002)). There is a continuing need for improved methods to discover natural product metabolites and genomic analysis of microorganisms provides a basis for the discovery of microbial secondary product metabolites.

High-throughput screening methods have been developed for the purpose of small molecule discovery for new drug candidates. The conventional high-throughput screening methods rely on trial-and-error methodologies, and there is a great deal of wasted effort in screening compounds without conducting pre-selection processes. Also, although there is a great deal of genomic information available and there continues to be more sequencing efforts undertaken, there is dearth of information linking genomic information to products of secondary metabolism. Where drug discovery efforts involve genomic analysis, such discovery methods often require time consuming and laborious steps required to identify the structure of the target metabolite. It is desirable to provide a method and system for identifying metabolic products from microorganisms that can be conducted on a high-throughput basis, and allows a high level of predictability based on genomic information.

### SUMMARY OF THE INVENTION

It is an object of the present invention to obviate or mitigate at least one disadvantage of the prior art. In certain embodiments of the invention, one or more of the following advantages are realized. The method and knowledge repository include a predictive aspect derived from previously obtained data. This allows the invention to traverse the “trial-and-error” style repetition normally associated with high throughput applications. Further, the invention advantageously incorporates knowledge of a microorganism's response to varying culture conditions (ingredients, temperature, osmotic pressure, etc), which allows prediction of conditions that may induce expression of a cryptic pathway. Feedback of secondary metabolite information to the knowledge repository gives the system efficiency, and increases the predictive power of the invention. In certain embodiments, linking of genetic capacity of a microorganism to produce a secondary metabolite of a particular chemical family lends efficiency if a compound of a specific chemical family is sought in the discovery process.

In one aspect, the invention provides a method of identifying a secondary metabolite synthesized by a target gene cluster contained within the genome of a microorganism, which method comprises the steps of: a) providing a microorganism containing a target gene cluster, wherein a putative or confirmed function has been attributed to at least one region of a gene in the gene cluster; b) obtaining from the microorganism an extract containing the secondary metabolite synthesized by the target gene cluster; c) measuring one or more chemical, physical or biological properties of metabolites in the extract; and d) identifying from the metabolites of step c) the secondary metabolite synthesized by the target gene cluster by comparing the chemical, physical or biological properties measured in step c) with the expected chemical, physical or biological properties of the secondary metabolite synthesized by the target gene cluster based on the putative or confirmed function attributed to the genes contained in the gene cluster. In one embodiment of this aspect, step b) involves growing the microorganism under multiple culture conditions to achieve expression of the target gene cluster and obtaining an extract of the fermentation broth produced under at least some of the culture conditions, and step c) involves measuring chemical, physical or biological properties of the metabolites of at least some of the extracts. In another embodiment of this aspect, step d) further comprises the step of comparing the chemical, physical or biological properties measured in step c) with the chemical, physical or biological properties of known compounds. In another embodiment of this aspect, step a) involves selecting a microorganism by reference to a knowledge repository containing information pertaining to at least one secondary metabolic gene cluster present in the genome of a microorganism. In another embodiment of this aspect, step b) involves growing the microorganism under multiple culture conditions selected by reference to a knowledge repository containing information pertaining to the culture conditions under which the product of at least one secondary metabolic gene cluster is expressed. In another embodiment of this aspect, step d) is under computer control with a knowledge repository containing information pertaining to metabolites synthesized by secondary metabolic gene clusters. In another embodiment of this aspect, step c) involves measuring one or more properties selected from the group consisting of molecular mass, UV spectrum and bioactivity. In another embodiment, the method includes a step of testing the secondary metabolite produced by the target gene cluster for biological activity, in particular antimicrobial, antifungal or anticancer activity. In another embodiment of this aspect, information pertaining to the association between the secondary metabolite and the target cluster; the chemical, physical or biological properties of the secondary metabolite; and the conditions under which the microorganism produces the secondary metabolite is added to a knowledge repository.

In a further aspect, the invention provides a method of identifying a secondary metabolite from a pre-selected chemical family comprising the steps of: a) establishing a correlation between the pre-selected chemical family, a structural feature of the secondary metabolite and a target gene cluster, wherein a putative or confirmed function has been attributed to at least one region of a gene in the gene cluster; b) selecting a microorganism containing the target gene cluster; c) obtaining from the microorganism an extract containing the secondary metabolite synthesized by the target gene cluster; d) measuring chemical, physical or biological properties of the metabolites in the extract; and e) identifying from the metabolites of step d) the secondary metabolite from the pre-selected chemical family by comparing the chemical, physical or biological properties of the secondary metabolite with the expected chemical, physical or biological properties based on the correlation between the pre-selected chemical family, the structural features of the secondary metabolite and the putative or confirmed function attributed to the genes contained in the gene cluster.

In a further aspect, the invention provides a system for identifying a secondary metabolite synthesized by a target gene cluster contained within the genome of a microorganism, said system comprising: a) genomic data indicating the presence of target gene cluster within a microorganism, wherein a putative or confirmed function has been attributed to at least one region of a gene in the gene cluster; b) extraction means for obtaining an extract derived from the microorganism, said extract containing metabolites comprising the secondary metabolite synthesized by the target gene cluster; c) an analyser for measuring chemical, physical or biological properties of metabolites in the extract; and d) a comparator for identifying from the metabolites contained in the extract the secondary metabolite synthesized by the target gene cluster by comparing the chemical, physical or biological properties measured by the analyser with the expected chemical, physical or biological properties of the secondary metabolite synthesized by the target gene cluster based on the putative or confirmed function attributed to the genes contained in the gene cluster. In another embodiment of this aspect, the invention provides a system for identifying a secondary metabolite from a pre-selected chemical family, the system comprising: a) genomic data establishing a correlation between the pre-selected chemical family, a structural feature of the secondary metabolite and a target gene cluster, wherein a putative or confirmed function has been attributed to at least one region of a gene in the gene cluster; b) a selector for selecting a microorganism containing the target gene cluster; c) extraction means for obtaining from the microorganism an extract containing the secondary metabolite synthesized by the target gene cluster; d) an analyser for measuring chemical, physical or biological properties of the metabolites in the extract; and e) a comparator for identifying from the metabolites analysed by the analyser the secondary metabolite from the pre-selected chemical family by comparing the chemical, physical or biological properties of the secondary metabolite with the expected chemical, physical or biological properties based on the correlation between the pre-selected chemical family, the structural features of the secondary metabolite and the putative or confirmed function attributed to the genes contained in the gene cluster.

In a further aspect, the invention provides a knowledge repository housing secondary metabolism data from a microorganism for identifying a secondary metabolite synthesized by a target gene cluster contained within the genome of a microorganism, said repository comprising: a) genomic data confirming the presence of a target gene cluster within a microorganism, wherein putative or confirmed function has been attributed to at least one region of a gene in the gene cluster; b) extract characterizing data providing chemical, physical or biological properties of metabolites contained in an extract derived from the microorganism, wherein said metabolites include a secondary metabolite attributable to the target gene cluster; and c) comparative data representing expected chemical physical or biological properties of the secondary metabolite synthesized by the target gene cluster, said extract characterizing data being comparable with the comparative data for identifying from the metabolites in an extract the secondary metabolite synthesized by the target gene cluster based on the putative or confirmed function attributed to said at least one region of a gene in a gene cluster. In another embodiment of this aspect, the knowledge repository additionally comprising culture conditions data linked to the extract characterizing data, the culture conditions data identifying culture conditions under which a set of extract characterizing data are obtained. In another embodiment of this aspect, the comparative data in the knowledge repository comprises a known compound library holding data characterizing a chemical, physical, or biological property of a plurality of known compounds for comparison with the extract characterizing data. In another embodiment of this aspect, a prediction link is made between a record within the genomic data and a record in the comparative data when a match is established between a secondary metabolite attributable to the target gene cluster within the extract characterizing data and the comparative data. In another embodiment of this aspect, the extract characterizing data of the knowledge repository comprises the biological property of antimicrobial, antifungal or anticancer activity. In another embodiment of this aspect, the knowledge repository of additionally comprising chemical family data linked to the genomic data assigning a chemical family to genomic data indicative of a putative or confirmed function in secondary metabolic pathways leading to synthesis of a member of the chemical family.

In a further aspect, the invention provides a method of building a knowledge repository housing secondary metabolism data from a microorganism for identifying a secondary metabolite synthesized by a target gene cluster contained within the genome of a microorganism, said method comprising the steps of: a) assembling genomic data confirming the presence of a target gene cluster within a microorganism, wherein putative or confirmed function has been attributed to at least one region of a gene in the gene cluster; b) inputting extract characterizing data providing chemical, physical or biological properties of metabolites observed in an extract derived from the microorganism, wherein said metabolites include a secondary metabolite attributable to the target gene cluster; and c) comparing the extract characterizing data with comparative data representing expected chemical physical or biological properties of the secondary metabolite synthesized by the target gene cluster, so as to identify from the metabolites in an extract the secondary metabolite synthesized by the target gene cluster based on the putative or confirmed function attributed to said at least one region of a gene in a gene cluster; and d) retaining the result of step c) by linking a secondary metabolite identified in the comparing step with the genomic data assembled in the assembling step. In another embodiment of this aspect, the invention provides a method of building a knowledge repository wherein the step of inputting extract characterizing data additionally comprises inputting culture conditions under which an extract is derived, and the step of retaining the result additionally comprises linking culture conditions to both the secondary metabolite identified in the comparing step and the genomic data assembled in the assembling step. In another embodiment of this aspect, the invention provides a method of building a knowledge repository wherein the step of inputting extract characterizing data comprising inputting the biological property of antibacterial, antifungal or anticancer activity.

In another embodiment of this aspect, the invention provides a method of building a knowledge repository housing secondary metabolism data from a microorganism for predicting secondary metabolite production from a target gene cluster based on genomic data, said method comprising: a) assembling genomic data confirming the presence of a target gene cluster within a microorganism, wherein putative or confirmed function has been attributed to at least one region of a gene within the gene cluster; b) extracting a medium containing said microorganism, thereby forming an extract; c) screening the extract for extract characterizing data indicative of the presence or absence of a secondary metabolite attributable to the target gene cluster based on a pre-selected chemical, physical or biological property; d) entering the extract characterizing data into the knowledge repository; e) comparing the extract characterizing data with comparative data representing expected chemical physical or biological properties of a secondary metabolite synthesized by the target gene cluster, so as to identify from the extract a secondary metabolite synthesized by the target gene cluster based on the putative or confirmed function; f) determining the identity of a secondary metabolite extracted; and g) affirming within the knowledge repository a correspondence between genomic data, the pre-selected chemical, physical or biological property, and the identity of the secondary metabolite, allowing a cycle of prediction of secondary metabolite production based on genomic data.

In a further aspect, the invention provides a memory for storing secondary metabolism data for access by an application program being executed on a data processing system for identifying a secondary metabolite synthesized by a target gene cluster contained within the genome of a microorganism, said memory comprising: a data structure stored in said memory, the data structure including information resident in a database used by said application program and including: genomic data confirming the presence of a target gene cluster within a microorganism, wherein putative or confirmed function has been attributed to at least one region of a gene in the gene cluster; extract characterizing data providing chemical, physical or biological properties of metabolites contained in an extract derived from the microorganism, wherein said metabolites include a secondary metabolite attributable to the target gene cluster; and comparative data representing expected chemical physical or biological properties of the secondary metabolite synthesized by the target gene cluster, said extract characterizing data being comparable with the comparative data for identifying the metabolites in an extract containing the secondary metabolite synthesized by the target gene cluster based on the putative or confirmed function attributed to said at least one region of a gene in a gene cluster.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached figures.

FIG. 1a is a schematic illustration of a general method and system for identifying secondary metabolites according to one embodiment of the invention. FIGS. 1 b, 1c, 1d, 1e, 1f and 1g illustrate the general method and systems of the FIG. 1a as described in examples 1, 2, 3, 4, 5, and 6 respectively.

FIG. 2 is a schematic illustration of a genomics-guided expression means to obtain from a microorganism extracts containing secondary metabolites and a genomics-guided screening technology to measure biological properties of the metabolites according to one embodiment of the invention.

FIG. 3 illustrates a high-throughput CHUMB method to obtain chemical, physical and biological properties of metabolites used in one embodiment of the invention.

FIG. 4 is a schematic illustration of a representative genomics-guided expression and screening technology to identify a metabolite according to one embodiment of the invention.

FIG. 5 is a schematic illustration of a representative genomics-guided extraction technology to isolate a metabolite according to one embodiment of the invention.

FIGS. 6, 7 and 8 are schematic illustration of a representative genomics-guided three-stage extraction/isolation/structure-elucidation protocol according to one embodiment of the invention; wherein Stage I of the protocol is shown in FIG. 6, Stage II of the protocol is shown generally in FIG. 7 (one example of the Stage II protocol of FIG. 7 is also shown in FIG. 6), and Stage II of the protocol is shown in FIG. 8.

FIG. 9 illustrates a schematic representation of a system for identifying a secondary metabolite synthesized by a target gene cluster.

FIG. 10 illustrates a schematic representation of a system for identifying a secondary metabolite from a pre-selected chemical family.

FIG. 11 illustrates a schematic representation of a typical graphical user interface according to the invention.

FIGS. 12a and 12b illustrate the results of a biochemical induction assay to detect enediyne metabolites based on their ability to damage DNA wherein, in FIG. 12a, CALI is calicheamicin, MACR is macromomycin, DYNE is dynemicin, and NEOC is neocarzinostatin, and in FIG. 12b, 007A is the putative enediyne from Amycolatopsis orientalis, 009C is the putative enediyne from Streptomyces ghanaensis, 145B is the putative enediyne from Streptomyces citricolor, and 046E and 171B are putative enediynes from the microorganisms in Ecopia's private culture collection.

FIG. 13 illustrates a graphical depiction of the 024A locus, a putative lipopeptide biosynthetic locus from Streptomyces refuineus, showing at the top of the figure, a scale in base pairs, followed by the coverage of the 024A locus in a single contiguous DNA sequence, the relative position and orientation of the 16 open reading frames (ORFs) forming the locus, indicating in black the unusual C-domain in the NRPS system (ORF 4) of the 024A locus, and finally the structural similarities between the lipopeptide synthesized by 024A (024A compound) and the known lipopeptide A54145 produced by Streptomyces fradiae.

FIGS. 14a and 14b are photographs of plates generated during extraction of an anionic lipopeptide from Streptomyces fradiae, and Streptomyces refuineus NRRL 3143 respectively, both showing an enrichment of activity based on IRA67 anion exchange chromatography consistent with expression of an acidic lipopeptide.

### DETAILED DESCRIPTION

The invention relates to an integrated genomics-based discovery platform designed to increase the rate at which products of secondary metabolism are discovered. The approach combines the technologies of traditional metabolite purification and isolation processes with genomic and bioinformatics technologies to identify compounds that are likely to have escaped detection in the past. The invention is genomics-based, and advantageously uses genomic information regarding a target gene cluster involved in a secondary metabolism pathway to predict the chemical, physical and biological properties of the metabolite produced by the target gene cluster, and in some embodiments to further assist in one or more of the following: selection of a target gene cluster or metabolite of interest; selection of a microorganism; and selection of culture conditions under which to grow the microorganism. The invention is computer-assisted and employs bioinformatics techniques. The invention is high-throughput, which allows expedited discovery in a convenient and efficient format. Further, the invention is iterative and the data generated in each iteration is fed back into the knowledge repository to strengthen the predictive and discovery capacity of the method.

A microorganism is provided or selected containing a target gene cluster involved in the synthesis of a secondary metabolite and for which target gene cluster there is genomic information. An extract from the microorganism is obtained which contains the secondary metabolite synthesized by the gene cluster. Chemical, physical or biological properties of metabolites present in the extract are assessed and compared with the chemical, physical or biological properties predicted to be associated with the metabolite based on the genomic information. Genomic-guided expression, screening and isolation is used to identify and isolate the metabolite synthesized by the target gene cluster.

The term “microorganism” refers to any prokaryotic or eukaryotic microorganism known or suspected to contain a gene cluster directed to the synthesis of a secondary metabolite. Bacteria and fungi are preferred microorganisms for use in the invention. Suitable bacterial species include substantially all bacterial species, both animal- and plant-pathogenic and nonpathogenic. Preferred microorganisms include but are not limited to bacteria of the order Actinomycetales, also referred to as actinomycetes. Preferred genera of actinomycetes include Nocardia, Geodermatophilus, Actinoplanes, Micromonospora, Nocardioides, Saccharothrix, Amycolatopsis, Kutzneria, Saccharomonospora, Saccharopolyspora, Kitasatosporia, Streptomyces, Microbispora, Streptosporangium, Actinomadura. The taxonomy of actinomycetes is complex and reference is made to Goodfellow (1989) Suprageneric classification of actinomycetes, Bergey's Manual of Systematic Bacteriology, Vol. 4, Williams and Wilkins, Baltimore, pp 2322-2339, and to Embley and Stackebrandt, (1994), The molecular phylogeny and systematics of the actinomycetes, Annu. Rev. Microbiol. 48, 257-289, for genera that may also be used with the present invention. In some embodiments, a knowledge repository is consulted to preferentially select a microorganism based on genomic information associated with a class of natural products, the presence of a target gene cluster, or production of a metabolite of interest.

The term “secondary metabolite” may be used interchangeably with the term “metabolite” and refers to a product arising from the biosynthesis involving a gene cluster within a microorganism which is a natural chemical product not normally employed in primary metabolic processes. The metabolite may be a member of a “chemical family” which is a grouping of chemical entities of natural products having a common physical attribute. Representative chemical families include polypeptides (including subgroups thereof such as lipopeptides and glycolipopeptides), terpenes, alkaloids, polysaccharides, enediynes, glycopeptides, orthosomycins, benzodiazepines, aminoglycosides, beta-lactams, amphenicols, lincosamides and polyketides (including subgroups thereof such as macrolides, ansamycins, glycosylated polyketides and aromatic polyketides). One skilled in the art would readily understand that a compounds having a polyketide backbone can be said to belong to the chemical family of “polyketides”, or that a compound having a polyene structure can be said to belong to the chemical family of “polyenes” etc. These exemplary chemical families should not be considered as limiting to the invention, as one skilled in the art could easily determine a desirable physical attribute of a chemical family of metabolites other than those exemplified herein.

The term target gene cluster refers to a gene, group of genes or a part of a gene involved in the biosynthesis of a secondary metabolite and for which there is genomic information. The term “target” is used simply to indicate that this is the particular gene cluster from which a metabolite of interest is expected to arise.

The term “genomic information refers to the nucleic acid sequence of a target gene cluster or amino acid sequence of the corresponding polypeptide(s), or both, together with functional annotation of the sequence information. The genomic information must be sufficient to provide a basis to make a prediction as to the chemical, physical or biological properties of the metabolite produced by a biosynthetic locus including the target gene cluster.

Many secondary metabolites are synthesized by a large multifunctional protein such as a nonribosomal peptide synthetase (NRPS) gene or a polyketide synthase (PKS) gene, and in such cases a “gene cluster” may be only part of a gene. Polyketides are synthesized by polyketide synthase (PKS) enzymes, which are complexes of multiple large proteins. Type 1 modular PKSs are formed by a set of separate catalytic active sites for each cycle of carbon chain elongation and modification in the polyketide synthesis pathway. Each active site is termed a domain. A set of active sites is termed a module. The typical modular PKS multienzyme system is composed of several large polypeptides, which can be segregated from amino to carboxy termini into a loading module, multiple extender modules, and a releasing module that frequently contains a thioesterase domain. Generally, the loading module is responsible for binding the first building block used to synthesize the polyketide and transferring it to the first extender module. The loading molecule recognizes a particular acyl-CoA and transfers it as a thiol ester to the ACP of the loading module. The AT on each of the extender modules recognizes a particular extender-CoA and transfers it to the ACP of that extender module to form a thioester. Each extender module is responsible for accepting a compound from a prior module, binding a building block, attaching the building block to the compound from the prior module, optionally performing one or more additional functions, and transferring the resulting compound to the next module. Each extender module contains a KS, AT, ACP, and zero, one, two or three domains that modify the beta-carbon of the growing polyketide chain. A typical (non-loading) minimal Type I PKS extender may contain a KS domain, an AT domain, and an ACP domain. Such domains are sufficient to activate a 2-carbon extender unit and attach it to the growing polyketide molecule. The next extender module, in turn, is responsible for attaching the next building block and transferring the growing compound to the next extender module until synthesis is complete. Once the PKS is primed with acyl-ACPs, the acyl group of the loading module is transferred to form a thiol ester (trans-esterification) at the KS of the first extender module; at this stage, extender module one possesses an acyl-KS and a malonyl- (or substituted malonyl-) ACP. The acyl group derived from the loading module is then covalently attached to the alpha-carbon of the malonyl group to form a carbon-carbon bond, driven by concomitant decarboxylation, and generating a new acyl-ACP that has a backbone two carbons longer than the loading building block (elongation or extension).

The polyketide chain, growing by two carbons with each extender module, is sequentially passed as covalently bound thiol esters from extender module to extender module, in an assembly line-like process. The carbon chain produced by this process alone would possess a ketone at every other carbon atom, producing a polyketone, from which the name polyketide arises. Most commonly, however, additional enzymatic activities modify the beta keto group of each two-carbon unit just after it has been added to the growing polyketide chain but before it is transferred to the next module.

In addition to the typical KS, AT, and ACP domains necessary to form the carbon-carbon bond, a module may contain other domains that modify the beta-carbonyl moiety. For example, modules may contain a ketoreductase (KR) domain that reduces the keto group to an alcohol. Modules may also contain a KR domain plus a dehydratase (DH) domain that dehydrates the alcohol to a double bond. Modules may also contain a KR domain, a DH domain, and an enoylreductase (ER) domain that converts the double bond product to a saturated single bond. An extender module can also contain other enzymatic activities, such as, for example, a methylase or dimethylase activity.

After traversing the final extender module, the polyketide encounters a releasing domain that cleaves the polyketide from the PKS and typically cyclizes the polyketide. The polyketide can be further modified by tailoring enzymes; these enzymes add carbohydrate groups or methyl groups, or make other modifications, i.e. oxidation or reduction, on the polyketide core molecule. Domains include ketosynthase (KS), acyl transferase (AT), acyl carrier protein (ACP), dehydratase (DH), ketoreductase (KR), enoylreductase (ER) etc. The order in which individual domains appear in a given polypeptide can be represented as “domain strings” that are characteristic signatures of such multidomain polypeptides such as PKS systems, non-ribosomal peptide synthetases (NRPSs) as well as hybrid PKS/NRPS systems. Given the specificity as to domains and modules in multimodular proteins, a “gene cluster” as used herein may refer to part of gene representing one or more domains or one or more modules of a multimodular system. Similarly “genomic information”, as used herein may refer to genomic information pertaining only to part of gene.

In other embodiments the genomic information relates to a group of genes involved in the biosynthesis of a characteristic moiety of a natural product metabolite. In still other embodiments, the genomic information relates to the full-length biosynthetic locus producing a metabolite, or several partial or full-length loci each producing a metabolite of a single class of natural products. The genomic information may be functional annotation of the gene cluster established by experimental results or a putative function attributed to the gene cluster by computer-assisted sequence comparison with the sequence of other known genes.

Genomic information may be obtained from a knowledge repository of genomic information which may be a computer database wherein the genomic information is electronically recorded and annotated with information available from public sequence databases such as GenBank National Center for Biotechnology lnfomration, NCBI and the Comprehensive Microbial Resource database (The Institute for Genomic Research). Alternatively genetic information may be generated according to any method known in the art such as methods employing nucleic acid probes, transposon-tagging, mutagenesis etc. Genetic information may also be generated by full genome sequencing of a microorganism. Another method that may be used to generate the genomic information is the high-throughput method for discovery of gene clusters described in CA 2,352,451 and U.S. Ser. No. 10/232,370 which advantageously provides a means to identify cryptic gene clusters, i.e. clusters of genes found in the genome of a microorganism and involved in the biosynthesis of a natural product metabolite which the microorganism has not previously been reported to produce. A cryptic gene cluster or biosynthetic locus containing a cryptic gene cluster may be expressed when the microorganism containing the cryptic gene cluster is grown under a particular set of culture conditions which may or may not be established. In some embodiments, the genomic information relates to a metabolite reported to be produced by a microorganism but for which the structure of the metabolite has not been elucidated.

The expression “chemical, physical or biological properties” refers to properties of a metabolite that are predicted based on the genomic data and subsequently measurable on a high throughput basis according to the invention. By “chemical property” is meant any chemical attributes or feature, such as the chemical structure, or the core structure, substructure or moiety of the metabolite of interest, or any chemical substituent, functionality or linkage found in the metabolite of interest. For example, the macrolide lactone ring structure of rosaramicins, the heterocyclic ring structure of benzopiazepines, the chromophore of enediynes, the amino acid residues of a peptide metabolite, the sugar residues in an oligosaccharide chain of a metabolite, the orthoester linkages of orthosomycins, the N-acyl peptide linkage of lipopeptides, the polyketide core structure of piericidins or dorrigocins would all be considered chemical properties of those respective metabolites of interest. By “physical property” is meant any measurable physical observations of a metabolite, including but not limited to molecular mass, UV spectrum. By “biological property” is meant the bioactivity or biological activity of a metabolite. “Bioactivity” and “biological activity” used herein with reference to a metabolite may be used interchangeably to refer to any observable activity possessed by the metabolite. Such activity may include, but is not limited to, antibacterial (gram-positive and/or gram negative), antifungal, anticancer, apoptotic or antiapoptotic activity or cell damaging activity as well as antiviral, immunosuppressant, hypocholesteremic, antihelmintic (e.g. cestodes, nematodes, schistosomes, trematodes), antiparasitic and insecticidal activities. Testing for such bioactivity or biological activity may be conducted using such tests as are known to those of skill in the art. For example, to test for antibacterial or antifungal activity, the effect of the metabolite on survival of a bacteria or fungus is evaluated. Similarly, anticancer, apoptotic, antiapoptotic, or other observable activities can be evaluated by exposing cells to the metabolite under conditions conducive to a particular activity to be countered. A biological induction assay (BIA) may be used to detect agents that damage DNA. The expression of chemical, physical or biological properties may refer to a single property—whether a chemical property, a physical property or a biological property—, or a combination of two or more properties—whether chemical properties, physical properties, biological properties, or a combination of chemical, physical and/or biological properties.

The invention uses genomics-guided expression, screening, isolation and structure elucidation technologies to identify the metabolite of interest from a target gene cluster. The expression “genomics-guided” refers to methods for expression, screening and isolating metabolites which find a basis in genomic information. By using genomics to guide such decisions as which microbe to investigate or which culture conditions to utilize in order to achieve synthesis of a metabolite, the random nature of high-throughput screening is traversed. Previous processes using high-throughput screening have not been guided by genetic information, but instead have been guided by such factors as the outcome of biological activity tests (for example, antimicrobial activity). In such cases of high-throughput screening where genomic information is not used, such biological activity tests are conducted on a very large number of products, but few if any will show efficacy. By guiding initial selection of a microbe, or other decisions such as culture conditions or isolation protocols and structure elucidation protocols on the basis of the genomic information that indicates that a microorganism has the ability to produce a secondary metabolite of interest, the number of samples that must be tested in order to obtain positive biological activity outcomes in high-throughput screening tests can be greatly reduced, and the efficiencies of the expression/screening processes are improved. The invention provides methods in which the genomic potential of a microorganism is considered, based on the presence of a target gene cluster within the genome of the microorganism. These methods are thus said to be genomics guided.

The term “extract” refers to a medium or fermentation broth in which a microorganism is cultured, or which is obtained from disrupting or otherwise deriving metabolites from a cell culture following an incubation period. In some embodiments, the extract is obtained by culturing the microorganism under culture conditions based on a link in the knowledge repository that serves to predict the conditions under which the microorganism is likely to express the target gene cluster and synthesize a desired metabolite. In other embodiments the culture conditions are selected with reference to a knowledge repository containing a link between a class of natural products and the culture conditions under which microorganisms have been reported to synthesize a metabolite of that class. Where the genomic information is associated with a cryptic target gene cluster, the microorganism is induced to express the target gene cluster and to synthesize the corresponding metabolite by growing the microorganism under multiple culture conditions. Minor modifications in medium composition and culture conditions can have a major influence of the range of secondary metabolites produced by a microorganism. In some embodiments, the culture conditions are selected to maximize the probability that the natural product metabolite produced by each secondary metabolic pathway present in the genome of a microorganism is expressed. Any conditions related to culture growth may be varied and used in association with the invention, for example pH, temperature, medium composition, humidity, pressure, the addition of pleiotropic factors or signalling molecules, etc. Other environmental conditions commonly known to effect natural product production such as the addition of DNA damaging agents, selective antibiotics and/or exposure to radiation can be used in combination with screening to select for alternate or enhanced natural product production in this invention.

For ease of reference, exemplary culture conditions and aqueous media formulations referred to herein are assigned a two-letter designation used throughout the present description and figures. AA is a medium containing 10 g/l of glucose; 40 g/l of corn dextrin, 15 g/l of sucrose, 10 g/l of casein hydrolysate (N-Z Amine A), 1 g/l of magnesium sulfate (MgSO4.7H₂O), and 2 g/l of calcium carbonate (CaCO₃). AB is a medium containing 24 g/l of glycerol; 25 g/l of mannitol; 25 g/l of soluble starch; 5.84 g/l of glutamine; 1.46 g/l of arginine; 1 g/l of sodium chloride (NaCl); 1 g/l of potassium phosphate, monobasic (KH₂PO₄); 0.5 g/l of magnesium sulfate (MgSO₄.7H₂O); and 2 ml/l of trace element solution and wherein the trace element solution is prepared by dissolving the following in 100 ml deionized, distilled (dd)H₂O: 0.1 g of FeSO₄.7H₂O; 0.01 g of MnSO₄.H₂O; 0.01 g of CuSO₄.5H₂O; 0.01 g of ZnSO₄.7H₂O; and 1 drop of concentrated sulphuric acid (H₂SO₄) is added as a stabilizer. BA is a medium containing 15 g/l of soybean powder; 10 g/l of glucose; 10 g/l of soluble starch; 3 g of sodium chloride (NaCl); 1 g/l of magnesium sulfate (MgSO₄.7H₂O); 1 g/l of potassium phosphate, dibasic (K₂HPO₄); and 1 ml of trace element solution produced by dissolve the following in 100 ml ddH₂O: 0.1 g of FeSO₄.7H₂O; 0.8 g of MnCl₂.4H₂O; 0.7 g of CuSO₄.5H₂O; 0.2 g of ZnSO₄.7H₂O, and 1 drop of concentrated sulphuric acid (H₂SO₄) added as a stabilizer. CA is a medium containing 40 g/l potato dextrin; 15 g/l of cane molasses; 10 g/l of glucose; 10 g/l of casein hydrolysate (N-Z Amine A); 1 g/l of magnesium sulfate (MgSO₄.7H₂O); and 2 g/l of calcium carbonate (CaCO₃). CB is a medium containing 20 g/l of sucrose; 2 g/l of bacto-peptone; 5 g/l of cane molasses; 0.1 g/l of ferrous sulfate heptahydrate (FeSO₄. 7H₂O); 0.2 g/l of magnesium sulfate heptahydrate (MgSO₄. 7H₂O); 0.5 g/l of potassium iodide (KI); 5 g/l of calcium carbonate (CaCO₃). CI is a medium containing 20 g/l of glycerol; 20 g/l of dextrin; 10 g/l of fish meal; 5 g/l of bacto-peptone; 2 g/l of ammonium sulfate (NH₄)₂SO₄; and 2 g/l of calcium carbonate (CaCO₃). DA is a medium containing 20 g/l of potato dextrin; 10 g/l of cane molasses; 10 g/l of glucose; 10 g/l of glycerol; 5 g/l of soluble starch; 5 g/l of soybean flour; 5 g/l of corn steep solids; 3 g/l of calcium carbonate (CaCO₃); 1 g/l of phytic acid; 0.1 g/l of ferrous chloride (FeCl₂.4H₂O); 0.1 g/l of zinc chloride (ZnCl₂); 0.1 g/l of manganese chloride (MnCl₂.4H₂O); 0.5 g/l of magnesium sulfate (MgSO₄.7H₂O). DY is a medium containing 10 g/l of corn starch; 5 g/l of pharmamedia; 1 g/l of CaCO₃; 0.05 g/l of CuSO₄ 5H₂O; 0.0005 g/l of NaI. DZ is a medium containing 15 g/l of soluble starch; 5 g/l of glucose; 10 g/l of cane molasses; 10 g/l of fish meal; and 5 g/l of calcium carbonate (CaCO₃). EA is a medium containing 50 g/l of lactose; 5 g/l of corn steep solids; 5 g/l of glucose; 15 g/l of glycerol; 10 g/l of soybean flour; 5 g/l of bacto-peptone; 3 g/l of calcium carbonate (CaCO₃); 2 g/l of ammonium sulfate (NH₄)2SO₄; 0.1 g/l of ferrous chloride (FeCl₂.4H₂O); 0.1 g/l of zinc chloride (ZnCl₂); 0.1 g/l of manganese chloride (MnCl₂.4H₂O); 0.5 g/l of magnesium sulfate (MgSO₄. 7H₂O). ES is a medium containing 40 g/l of glucose; 5 g/l of dried yeast; 1 g/l of K₂HPO₄; 1 g/l of MgSO₄; 1 g/l of NaCl; 2 g/l of (NH₄)2SO₄; 2 g/l of CaCO₃; 0.001 g/l of FeSO₄ 7H₂O; 0.001 g/l of MnCl₂ 4H₂O; 0.001 g/l of ZnSO₄ 7H₂O; 0.0005 g/l of NaI. ET is a medium containing 60 g/l of molasses; 20 g/l of soluble starch; 20 g/l of fish meal; 0.1 g/l of copper sulfate (CuSO₄.5H₂O); 0.5 mg/l of sodium iodide (NaI); and 2 g/l of calcium carbonate (CaCO₃). FA is a medium containing 40 g/l of potato dextrin; 15 g/l of cane molasses; 10 g/l of glucose; 10 g/l of casein hydrolysate (N-Z Amine A); 3 g/l of sodium phosphate, dibasic, anhydrous (Na₂HPO₄); 1 g/l of magnesium sulfate (MgSO₄.7H₂O); and, after adjusting pH to 7.0, 2 g/l of calcium carbonate (CaCO₃). GA is a medium containing 103 g/l of sucrose; 10 g/l of glucose; 5 g/l of yeast extract; 0.1 g/l of casamino acids; 10.12 g/l of magnesium chloride (MgCl₂.6H₂O); and 0.25 g/l of potassium sulfate (K₂SO₄); and per litre of medium 10 ml of KH₂PO₄ (0.5% solution); 80 ml of CaCl₂.2H₂O (3.68% solution); 15 ml of L-proline (20% solution); 100 ml of TES buffer (5.73% solution, adjusted to pH 7.2); 5 ml of NaOH (1N solution); and 2 ml of trace element solution. HA is a medium containing 340 g/l of sucrose; 10 g/l of glucose; 5 g/l of bacto-peptone; 3 g/l of yeast extract; 3 g/l of malt extract; and 1 g/l of magnesium chloride (MgCI₂.6H₂O). IA is a medium containing: 40 g/l of soybean powder; 30 g/l of soluble starch; 20 g/l of glucose; 3 g/l of ammonium nitrate (NH₄NO₃); and, after adjusting pH to 6.2, 1 g/l of calcium carbonate (CaCO₃). IB is a medium containing 40 g/l of mannitol; 33 g/l of casein hydrolysate (N-Z Amine A); 10 g/l of yeast extract; 9 g/l of potassium phosphate, monobasic (KH₂PO₄); and 5 g/l of ammonium sulfate (NH₄)2SO₄. JA is a medium containing 35 g/l of malt extract; 30 g/l of corn starch; 15 g/l of corn steep liquor; 15 g/l of pharmamedia; and, after adjusting pH to 7.3, 2 g/l of calcium carbonate (CaCO₃). KA is a medium containing 10 g/l of glucose; 10 g/l of corn steep liquor; 10 g/l of soybean powder; 5 g/l of glycerol; 5 g/l of dry yeast; 5 g/l of sodium chloride (NaCl); and, after adjusting pH to 5.7, 2 g/l of calcium carbonate (CaCO₃). KC is a medium containing 40 g/l of tomato puree; 2 g/l of glucose; 15 g/l of oatmeal; 50 mcg/l of CoCl2.2H2O. KD is a medium containing 15 g/l of dextrin; 20 g/l of soluble starch; 10 g/l of soybean meal; 3 g/l of meat extract; 3 g/l of polypeptone; 3 g/l of yeast extract; 3 g/l of calcium carbonate; and 1 g/l of sodium chloride. KE is a medium containing 30 g/l of glycerol; 15 g/l of distiller's solubles; 10 g/l of pharmamedia; 10 g/l of fish meal; and 6 g/l of calcium carbonate (CaCO₃). KF is a medium containing 1 g/l of glucose; 24 g/l of soluble starch; 3 g/l of bacto peptone; 3 g/l of meat extract; 5 g/l of yeast extract; and 4 g/l of calcium carbonate. KG is a medium containing 10 g/l of bacto-peptone; 10 g/l of glucose; 20 g/l of cane molasses; 1 g/l of calcium carbonate; and 0.1 g/l of ferric ammonium citrate. LA is a medium containing 25 g/l of soluble starch; 15 g/l of soybean powder; 5 g/l of dry yeast; and 2 g/l of calcium carbonate (CaCO₃). MA is a medium containing 25 g/l of soluble starch; 15 g/l of soybean powder; 2 g/l of dry yeast; 5 g/l of sodium chloride (NaCl); 4 g/l of calcium carbonate (CaCO₃); and 2 g/l of ammonium sulfate (NH₄)2SO₄. MC is a medium containing 10 g/l of glucose; 10 g/l of starch; 15 g/l of soybean meal; 1 g/l of KH₂PO₄; 3 g/l of NaCl; 1 g/l of MgSO₄ 7H₂O; 0.007 g/l of CuSO₄ 5H₂O; 0.001 g/l of FeSO₄ 7H₂O; 0.008 g/l of MnCl₂ 4H₂O; 0.002 g/l of ZnSO₄ 5H₂O; MU is a medium containing 25 g/l of mannitol; 10 g/l of soybean powder; 10 g/l of beef extract; 5 g/l of bacto-peptone; 5 g/l of glucose; 2 g/l of sodium chloride (NaCl); 3 g/l of calcium carbonate (CaCO₃). NA is a medium containing 20 g/l of glycerol; 10 g/l of cane molasses; 5 g/l of caseamino acids; 1 g/l of bacto-peptone; 4 g/l of calcium carbonate (CaCO₃). NE is a medium containing 30 g/l of glucose; 5 g/l of bacto-peptone; 5 g/l of beef extract; 5 g/l of sodium chloride (NaCl); 2 g/l of calcium carbonate (CaCO₃). NF is a medium containing 20 g/l of soluble starch; 20 g/l of soybean meal; 5 g/l of NaCl; 5 g/l of yeast extract; 2 g/l of CaCO₃; 0.005 g/l of MnSO₄; 0.005 g of CuSO₄; 0.005 g/l of ZnSO₄. NG is a medium containing 40 g/l glucose; 15 g/l of caseamino acids; 5 g/l of NaCl; 2 g/l of CaCO₃; 1 g/l of K₂HPO₄; 12.5 g/l of MgSO₄. OA is a medium containing 10 g/l of glucose; 5 g/l of glycerol; 3 g/l of corn steep liquor; 3 g/l of beef extract; 3 g/l of malt extract; 3 g/l of yeast extract; 2 g/l of calcium carbonate (CaCO₃); 0.1 g/l of thiamine. PA is a medium containing 10 g/l of soluble starch; 10 g/l of glycerol; 5 g/l of glucose; 5 g/l of beef extract; 3 g/l of bacto-peptone; 2 g/l of yeast extract; 1 g/l of casamino acids; 2 g/l of calcium carbonate (CaCO₃); 0.01 g/l of thiamine. PB is a medium containing 25 g/l of soybean meal; 7.5 g/l of soluble starch; 22.5 g/l of glucose; 3.5 g/l of dry yeast; 0.5 g of zinc sulfate (ZnSO₄.7H₂O); 6 g/l of calcium carbonate (CaCO₃). QB is a medium containing 10 g/l of soluble starch; 12 g/l of glucose; 10 g/l of Pharmamedia; 5 g/l of corn steep liquor; 4 ml/l of proflo oil. RA is a medium containing: 20 g/l of soluble starch; 5 g/l of pharmamedia; 2.5 g/l of yeast extract; 1 g/l of sodium chloride (NaCl); 0.75 g/l of potassium phosphate, dibasic (K₂HPO₄); 1 g/l of magnesium sulfate (MgSO₄.7H₂O); 3 g of calcium carbonate (CaCO₃). RB is a medium containing 60 g/l of corn starch; 15 g/l of linseed meal; 10 g/l of glucose; 5 g/l of yeast extract; 1 g/l of ferrous sulfate (FeSO₄.7H₂O); 1 g/l of ammonium sulfate (NH₄)2SO₄; 1 g/l of ammonium phosphate (NH4H2PO4); 10 g/l of calcium carbonate (CaCO₃). RC is a medium containing 10 g/l of corn dextrin; 10 g/l of bacto-tryptone; 10 g/l of molasses; 2 g/l of sodium chloride (NaCl); 5 g/l of calcium carbonate (CaCO₃). RM is a medium containing 100 g/l of sucrose; 0.25 g/l of K₂SO₄; 10.128 g/l of MgCl₂.6H₂O; 21 g/l of MOPS; 10 g/l of glucose; 0.1 g/l of casamino acids; 5 g/l of yeast extract; 2 ml/l of trace elements. KH is a medium containing: 10 g/l of glucose; 20 g/l of potato dextrin; 5 g/l of yeast extract; 5 g/l of NZ Amine A; and 1 g/l of Mississippi lime (substitute CaCO₃). SF is a medium containing 25 g/l of glucose; 18.75 g/l of soybean powder; 3.75 g/l of cane molasses; 1.25 g/l of casein hydrolysate (N-Z Amine A); 8 g/l of sodium acetate; and 3 g/l of calcium carbonate (CaCO₃). SM is a medium containing 5 g/l of glucose; 5 g/l of starch; 7.5 g/l of soybean powder; 0.5 g/l of K₂HPO₄; 1.5 g/l of NaCl; 0.5 g/l of MgSO₄; 0.500 ml/l of 1000× metal salts; and 500 ml/l of H₂O. SP is a medium containing 20 g/l of glucose; 5 g/l of bacto-peptone; 5 g/l of beef extract; 5 g/l of sodium chloride (NaCl); 3 g/l of yeast extract; and 3 g/l of calcium carbonate (CaCO₃). QB is a medium containing: 5 g/l of starch; 6 g/l of glucose; 2.5 g/l of corn steep liquor; 5 g/l of pharmamedia; 2 ml/l of proflo oil. TA is a medium containing 103 g of sucrose; 5 g of yeast extract; 0.1 g of caseamino acids; 10.12 g of magnesium chloride (MgCl₂.6H₂O); 0.25 g of potassium sulfate (K₂SO₄); and after autoclaving, 10 ml of KH₂PO₄ (0.5% solution); 80 ml of CaCl₂. 2H₂O (3.68% solution); 15 ml of L-proline (20% solution); 100 ml of TES buffer (5.73% solution, adjusted to pH 7.2); 5 ml of NaOH (1N solution); and 2 ml of trace element solution. VA is a medium containing 50 g/l of glucose; 30 g/l of soybean flour; 5 g/l of sodium chloride (NaCl); 3 g/l of ammonium sulfate (NH₄)2SO₄; and 6 g/l of calcium carbonate (CaCO₃). VB is a medium containing 20 g/l of sucrose; 20 g/l of cane molasses; 10 g/l of glucose; 5 g/l of soytone-peptone; and 2.5 g/l of calcium carbonate (CaCO₃). WA is a medium containing 0.8 g/l of yeast extract; 0.5 g/l of casamino acids; 0.4 g/l of glucose; 2 g/l of potassium phosphate, dibasic (K₂HPO₄). XA is a medium containing 10 g/l of yeast extract; 10 g/l of casein hydrolysate (N-Z Amine A); 5 g/l of beef extract; 3 g/l of magnesium sulfate (MgSO₄.7H₂O); and 1 g/l of potassium phosphate, dibasic (K₂HPO₄). YA is a medium containing 10 g/l of bacto-peptone; 8 g/l of beef extract; 3 g/l of yeast extract; 5 g/l of glucose; 5 g/l of lactose; 2.5 g/l of potassium phosphate, dibasic (K₂HPO₄); 2.5 g/l of potassium phosphate, monobasic (KH₂PO₄); 0.2 g/l of magnesium sulfate (MgSO₄.7H₂O); and 0.05 g/l of manganese sulfate (MnSO₄.H₂O). ZA is a medium containing 10 g/l of sucrose; 8 g/l of casein hydrolysate (N-Z Amine A); 4 g/l of yeast extract; 3 g/l of potassium phosphate, dibasic (K₂HPO₄); and 0.3 g/l of magnesium sulfate (MgSO₄. 7H₂O).

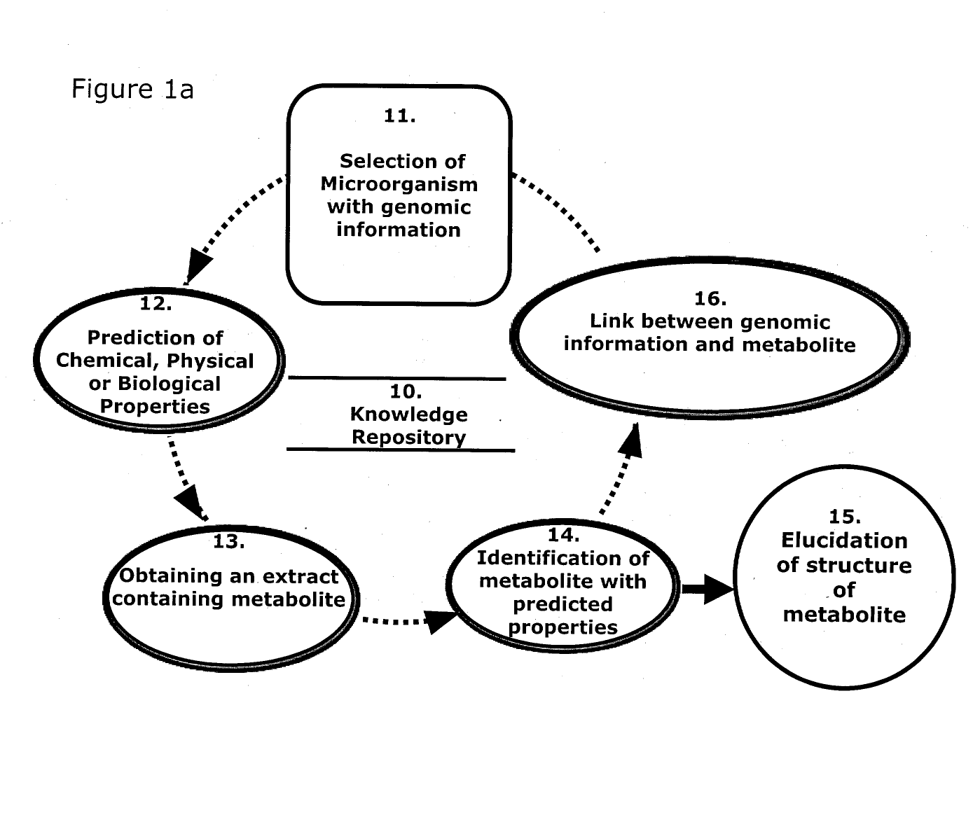

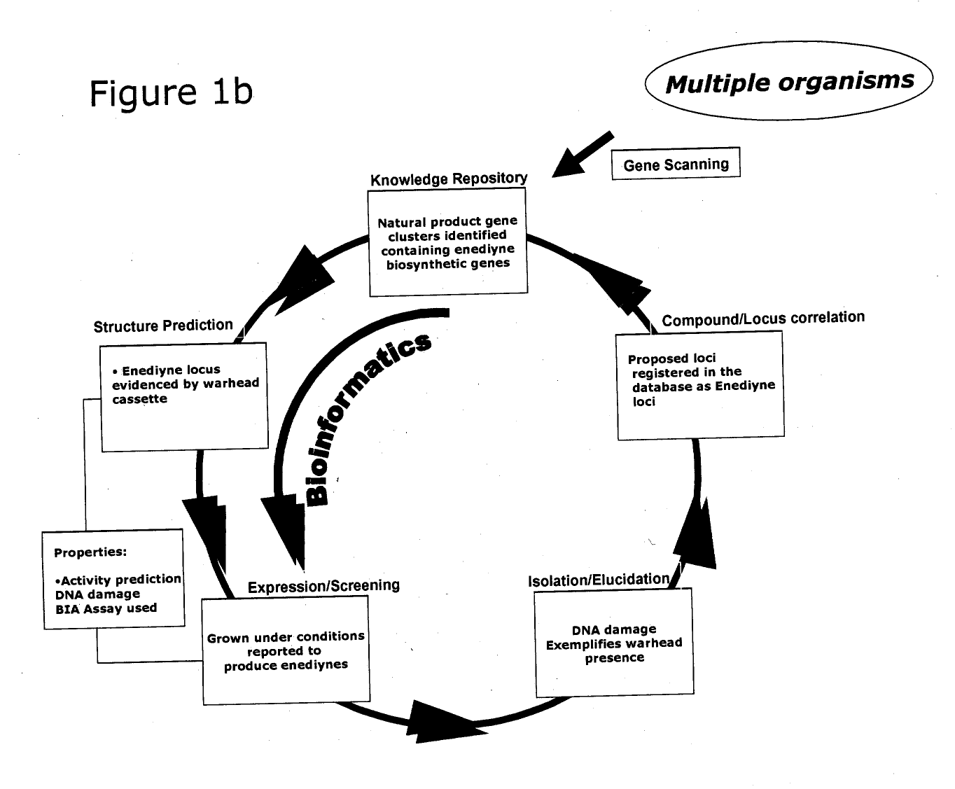

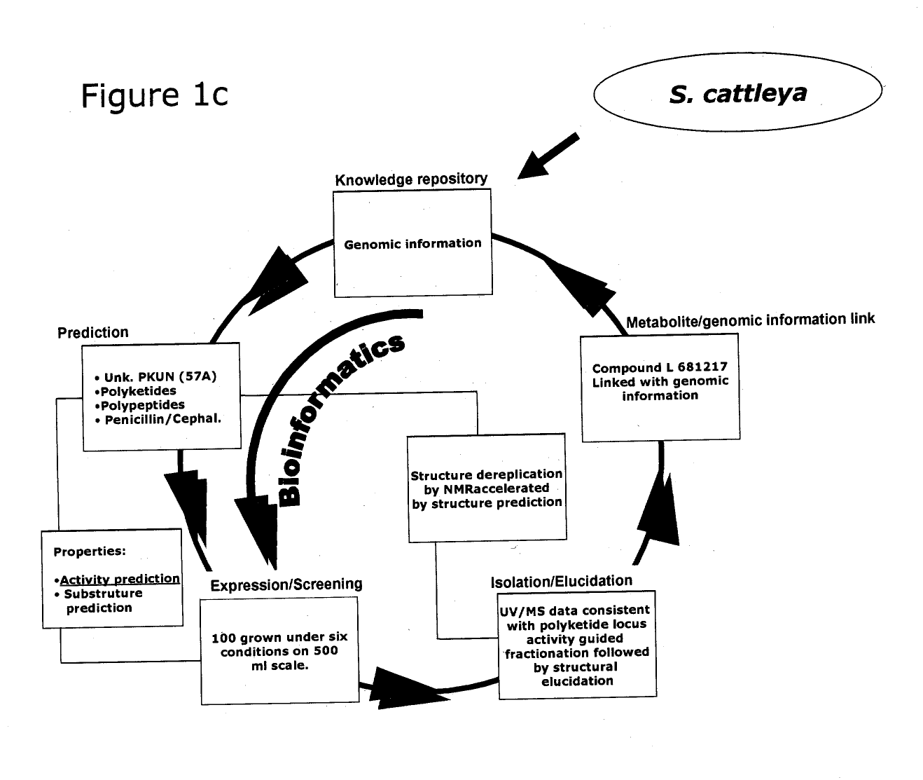

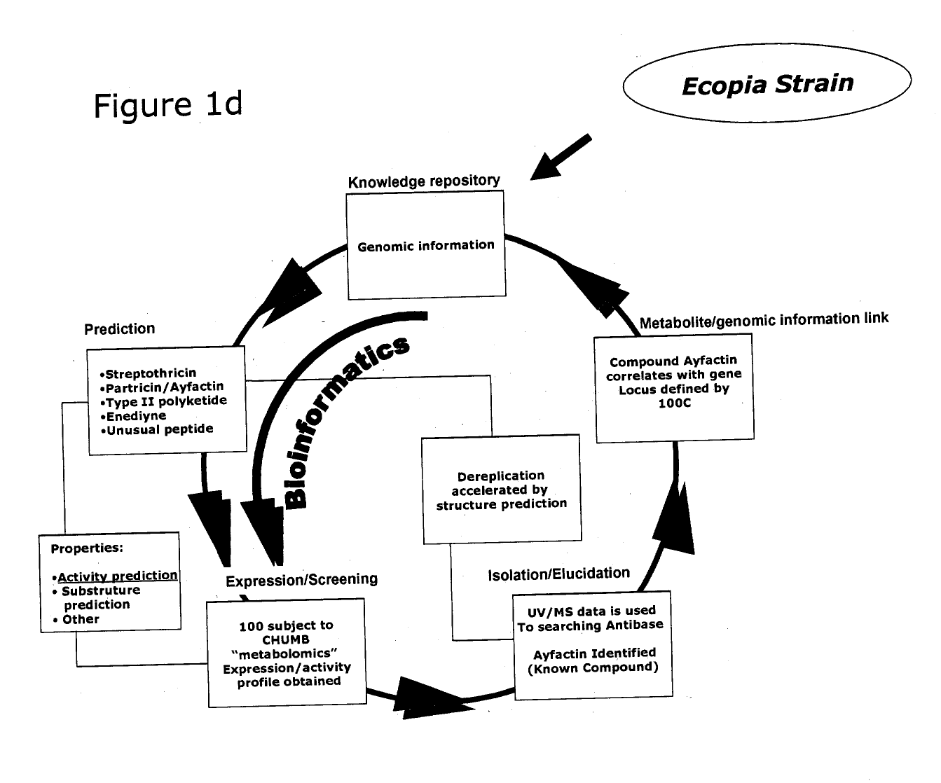

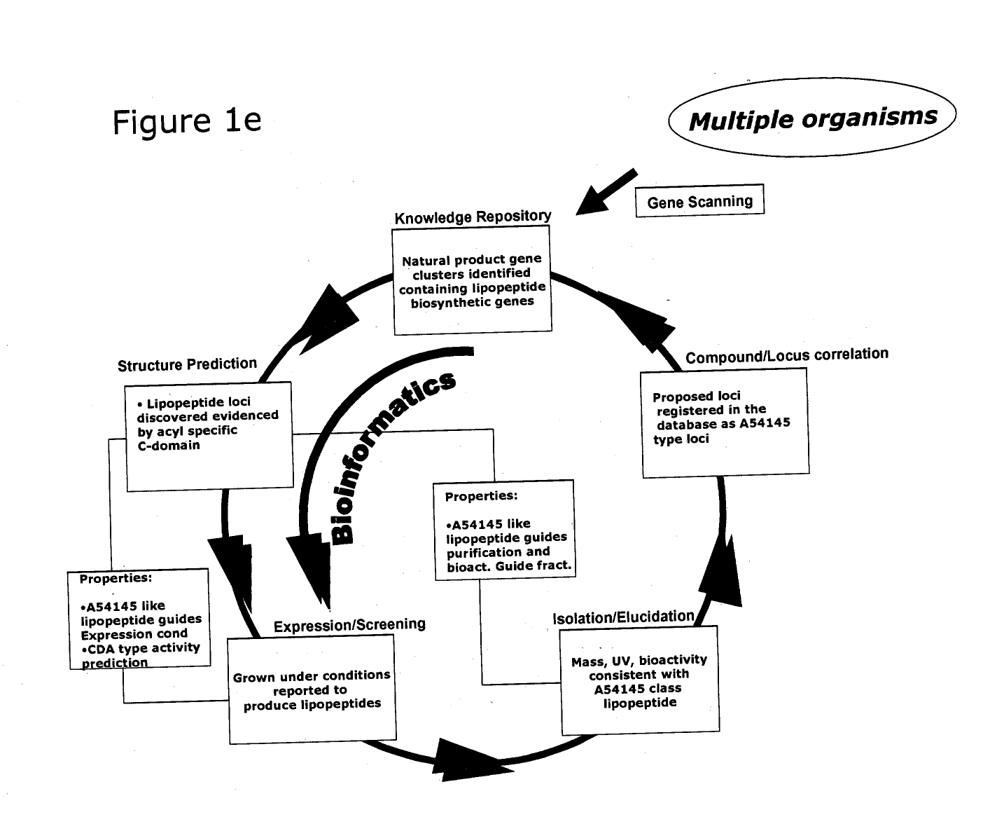

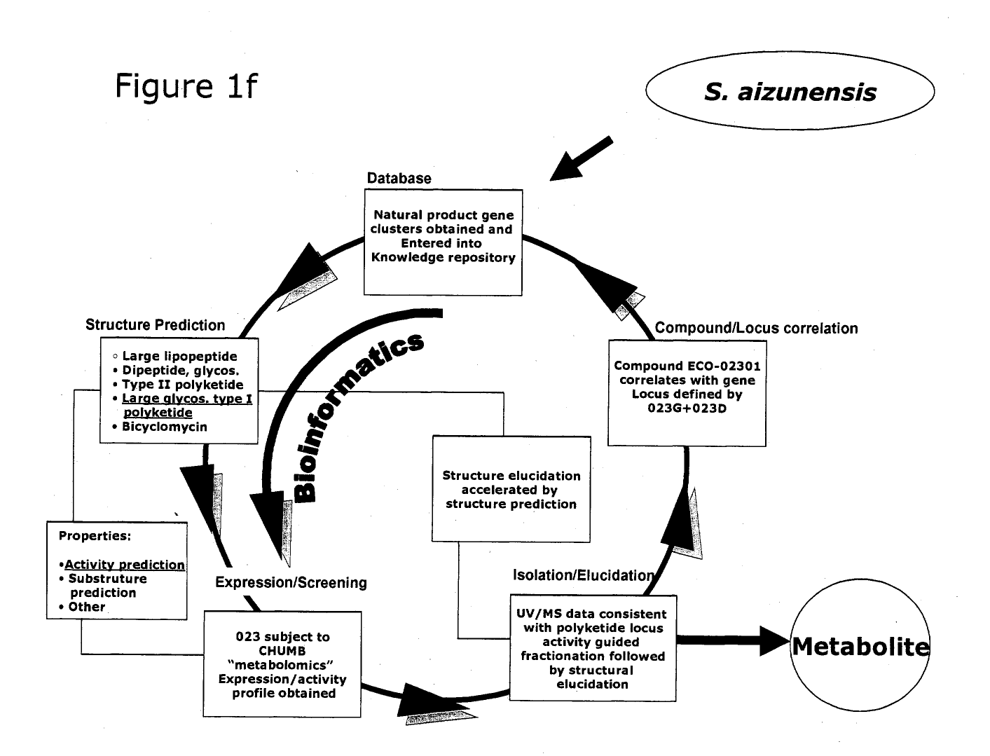

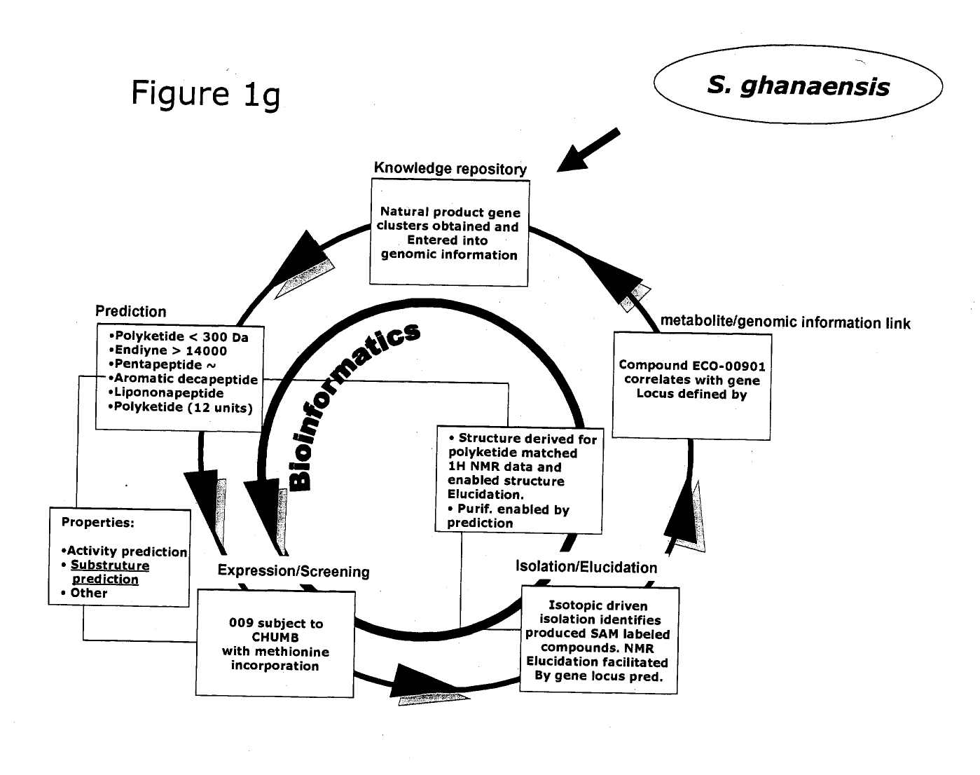

As illustrated in FIG. 1a, a microorganism (11) is selected. The microorganism contains a target gene cluster for which there is genomic information. The genomic information is used as a basis to make predictions (12) regarding chemical, physical or biological properties of the metabolite of interest. The predicted chemical, physical or biological properties direct the subsequent steps. The microorganism is induced to produce the metabolite synthesized by the target gene cluster and an extract with the metabolite of interest is obtained (13). Chemical, physical or biological properties of the metabolites in the extract are measured. The metabolite of interest is identified from the extract (14) by comparing the measured chemical, physical or biological properties with the predicted chemical, physical or biological properties of the metabolite of interest. A link (16) may be made in the knowledge repository between the metabolite and the target gene cluster. In some embodiments, the complete structure is elucidated (15) using genomic-guided methods. FIGS. 1b, 1c, 1d, 1e, 1f and 1g are embodiments of the method of FIG. 1a as described in each of examples 2, 3, 4, 5 and 6 respectively. FIG. 1b illustrates an embodiment where multiple metabolites of a pre-selected chemical family are identified. FIGS. 1c, 1d and 1f illustrate embodiments where the optional computer-assisted dereplication aspect of the invention is used. FIGS. 1c, 1d and 1f further illustrate embodiments where the optional structure elucidation step of the metabolite-of interest is performed. FIG. 1e illustrates an embodiment where the gene cluster is composed merely of part of a single gene. FIG. 1c illustrates an embodiment where a microorganism is randomly-selected and its genome is analyzed for the presence of cryptic gene clusters.

The invention is iterative and information generated during each iteration of the invention as well as links or associations between data elements established during each iteration of the invention may be fed back and stored into a knowledge repository to strengthen the predictive capacity of the invention. By way of example, in one embodiment, a link is made between the target gene cluster and the metabolite produced. In another embodiment a link is made between the metabolite produced and the microorganism selected. In a further embodiment a link is made between the genomic information and a chemical family. In a further embodiment a link is made between the culture conditions under which a microorganism is induced to synthesize a metabolite and the metabolite. In a further embodiment a link between chemical, physical and biological properties and a metabolite of interest. It is to be understood that the invention does not require any particular link to be created and stored in the knowledge repository in order that the method or system of the invention achieve its objective of identifying a secondary metabolites. However, various embodiments may include a step wherein any one or more of the above links are created, fed-back and stored in the knowledge repository.

The invention contemplates use of conventional expression, screening, isolation and structure elucidation technologies and one skilled in the art could readily select appropriate technologies for use with the invention having regard to any one or more of the following factors: the target gene cluster, the metabolite of interest, the chemical class of interest, the microorganism selected, the predicted chemical, physical and biological properties etc. Preferred expression, screening, isolation and structure elucidation technologies are high-throughput or genomics-guided or both high-throughput and genomics-guided. By way of example, an appropriate screening technology would allow for the use of a battery of assays. In one embodiment an antibiotic screening assay for use with the invention incorporates a multi-well plate format (for example, a 96-well plate) to increase throughput. In another embodiment, the screening technology selected allows for the simultaneous screening of thousands of fermentation broths for antimicrobial activities.

In some embodiments, genomics-guided biological screening steps may be used to identify the best candidates for a more time-consuming chemistry isolation process. For example, if the genomics information indicates that the microorganism contains a gene clusters producing a compound of a class known to have activity against certain set of indicator organisms (Gram-positive, Gram-negative or activity against a particular organism), then the bioassay results may be used to select appropriate broths or extracts for chemical analysis. Alternatively, if the genomics information indicates that a microorganism may produce a previously-identified compound with known activity against certain indicator organisms, then it may be desirable to disfavor extracts that display activity against those indicator organisms when selecting extracts for chemical analysis.

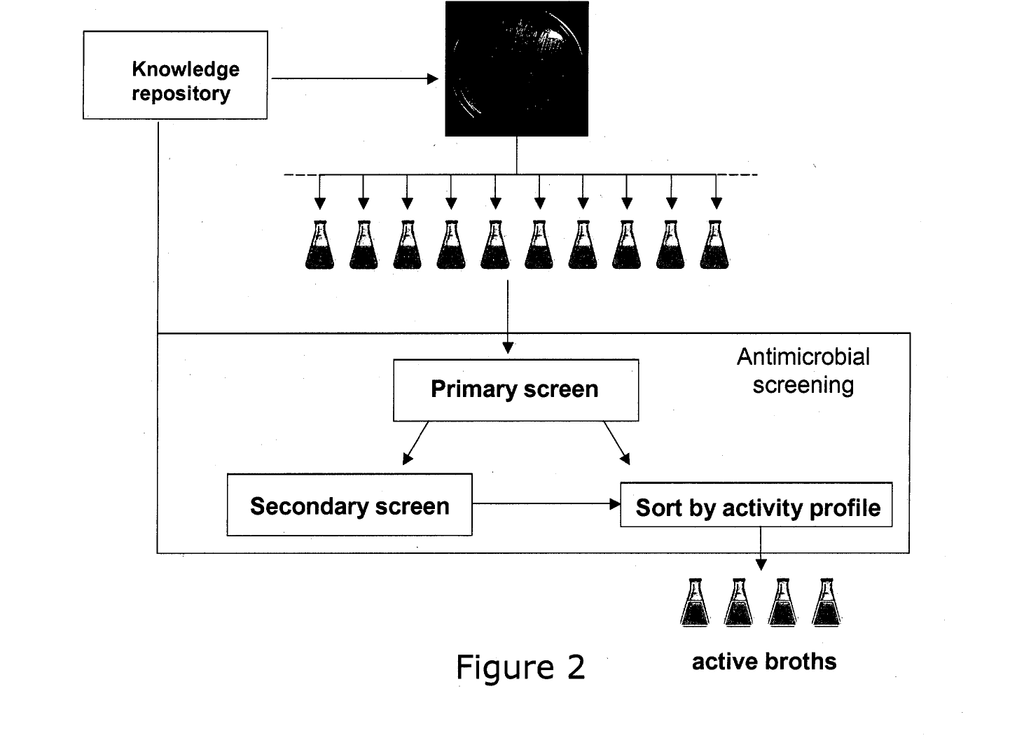

FIG. 2 illustrates one appropriate expression and screening technology for measuring biological properties of metabolites. In FIG. 2, extracts are screened against a panel of indicator microorganisms to identify metabolites with a particular biological activity. Extracts are tested for antibiotic activity against a panel of indicator strains, which may include bacterial (gram-positive and gram-negative) and fungal pathogens. Active extracts are sorted according to activity profile and representative extracts are selected for chemical analysis. In some embodiments, biological screening steps may be used to identify the best candidates for a more time-consuming chemistry isolation process.

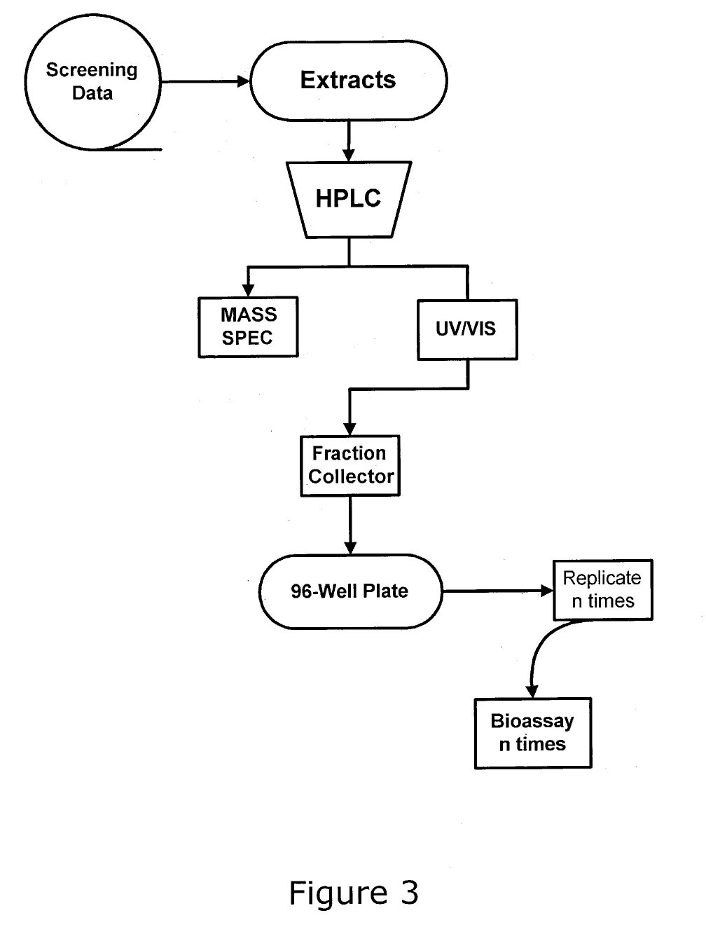

A convenient high-throughput protocol to assess chemical, physical and biological properties appropriate for use with the invention is referred to in the description and figures as CHUMB. As illustrated in FIG. 3, the CHUMB method fractionates extracts and generates data for each fraction in a given extract, including a UV trace by chromatographic mobility, a mass trace by chromatographic mobility providing the molecular weight of compounds in the fraction, and a bioactivity assessment of the compounds in the fraction, in a form which may readily be fed back to and stored in the knowledge repository. Using the CHUMB method, an extract is run through a chromatography column and is fractionated according to the mechanism of the chromatography media selected. For instance, a C-18 (octadecyl silane-functionalized silica gel) column run with an organic solvent gradient tends to separate compounds on the basis of their hydrophobicity. The output flow from the column is split with about 10% of flow provided for mass spectrometer analysis and about 90% flowing through a UV detector and then directed to a 96-well plate, fractionated by hydrophobicity. Bioactivity of the samples in the 96-well plate is assessed using one or more indicator strains or biological/biochemical assays to identify the bioactive fractions.

The metabolites produced by the target gene clusters are isolated from the samples of crude extract obtained from fermentation of a pure culture of the selected microorganism. Each sample would be expected to contain secondary metabolites exhibiting bioactivity against indicator strains, primary metabolites not generally exhibiting bioactivity against indicator stains, enzymes and fragments of enzymes involved in the biosynthesis of primary or secondary metabolic compounds, as well as biomass from media and whole cells. The crude extract is purified using known methods and guided by the a comparison of the measured chemical, physical and biological properties of the metabolites in each sample with the predicted chemical, physical and biological properties of the metabolite based on the genomic information to obtain purified samples containing single natural product metabolites. For example, the mass, UV and bioactivity of metabolites in each fraction may be compared with a database of known natural products in a dereplication step. A knowledge repository or database may be used in the dereplication step by comparing chemical, physical or biological data measured with the predicted chemical physical and biological properties based on genomic information from the microorganism used. Finally, the structure of the metabolite is solved, using well-known analytical methods, and the structure information fed back to and stored in the knowledge repository.

Genomics-based expression protocols employ conventional microbial growth fermentation methods, but give consideration to genomic information so as to make a rational selection regarding the culture conditions that will likely induce a microorganism to express a target gene cluster. One standard fermentation method that may be used is as follows. An agar plate of an appropriate medium is streaked with a glycerol stock of the desired organism and incubated at 30° C. for 2-7 days until colonies appear. The colonies are examined for contamination by microscopic analysis. Several loops of mycelia and/or spores are transferred to a sterile centrifuge tube along with a sterile medium (e.g. TSB medium), and crushed with a sterile centrifuge tube cell crusher. The crushed cell suspension is transferred to a sterile flask with appropriate seed culture medium (e.g. TSB), and 3 glass beads. The seed culture is shaken at about 250 rpm at 30° C. for 2-3 days until substantial cell density is present. Culture is again examined for contamination by microscopic analysis. For fermentation, about 25 to 500 mL of fermentation medium is prepared and sterilized in a large Erlenmeyer flask (125 ml to 4L). Two to ten ml of seed culture is added to an appropriate volume of culture medium in the fermentation flask and incubated at 30° C. for 2-7 days with shaking at 250 rpm. The culture is examined for contamination by microscopic analysis.

Samples of the fermentation broth from the culture conditions used are collected and chemical, physical or biological properties of the metabolites in the samples are measured. The chemical physical or biological properties may be assayed by using many conventional methods including but not limited to spectroscopic, chromatographic, or biological methods or assays. Spectroscopic characterization methods include mass spectrometry, UV spectroscopy, NMR spectroscopy, IR spectroscopy, and X-ray diffraction analysis. Chromatographic methods characterize compounds on the basis of their mobility, or the lack thereof, in chromatographic systems such as such size exclusion chromatography, adsorption chromatography, partition chromatography, hydrophobic interaction chromatography, ion-exchange chromatography, and affinity chromatography. Biological assays include, but are not limited to cell-based methods such as antibacterial, antifungal, antiviral, antiprotozoal or eukaryotic cell differentiation, metabolism or cytotoxicity assays; multicellular organism-based assays such as insecticidal or antihelmintic (e.g. cestodes, nematodes, schistosomes, trematodes etc.) assays; or in vivo/in vitro biological assays, such as enzyme inhibition, DNA damage detection, immunological assays, ligand binding or other biochemical assays. Isotopic precursor and precursor analog incorporation methods provide a ready access to precursor and product functionality. It is generally known that supplementing fermentation growth media with isotopically labeled precursors or precursor analogs results in the partial (0.05-60% or more) incorporation of such isotopically- or chemically-labeled precursors into secondary metabolites which are biosynthesized via said precursors. Such incorporation can be investigated by a variety of analytical methods including, but not limited to, radiometry (e,g, ¹⁴C, ³H, ³²P, ³⁵S incorporation for isotopically-radiolabeled precursors), mass spectrometry (for stable and unstable isotopically labeled precursors and precursor analogs), or NMR (for spin-active nuclides). Precursors may include, but are not limited to primary metabolites, secondary metabolic intermediates, and precursor analogs. Genomic information regarding a target gene cluster and the metabolite of interest in a given organism allows for labeled precursors to be rationally selected, supplemented into the growth media, and the cryptic products of fermentation to be detected and resolved on the basis of the properties of the isotope-enriched products.

The metabolites synthesized by the target gene cluster are isolated from fermentation broths by a series of isolation and extraction steps designed to compare the measured chemical, physical or biological properties of the metabolites in the samples and the predicted chemical, physical or biological properties based on the genomic information.

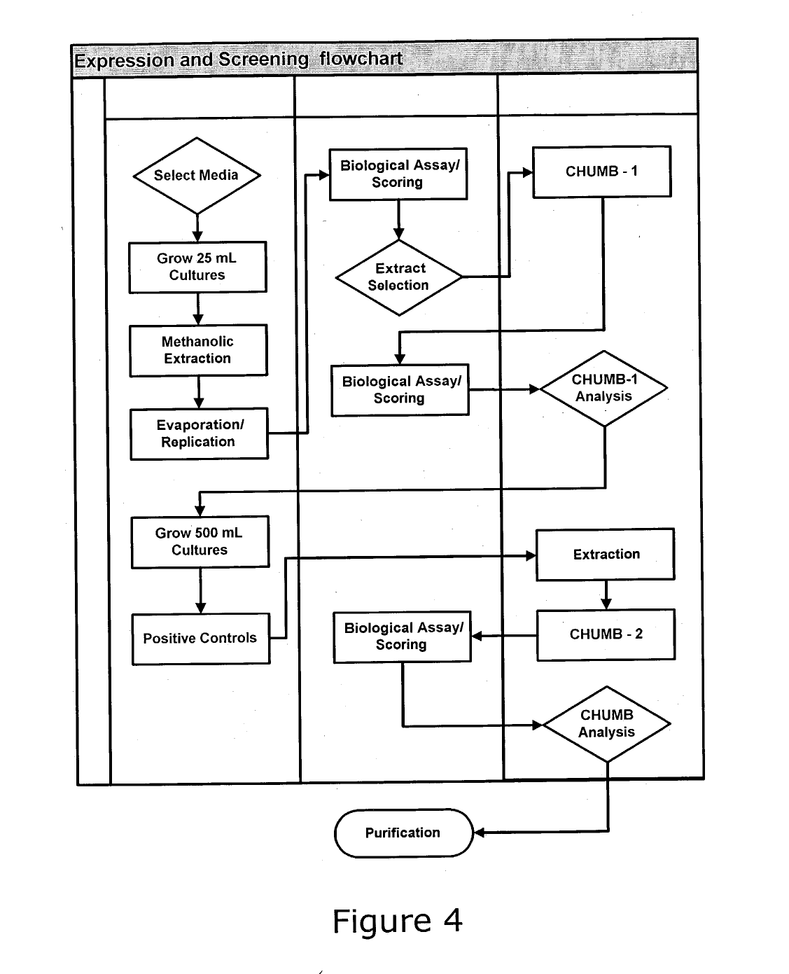

A representative genomics-guided expression and screening scheme for metabolite identification according to one embodiment of the invention is illustrated in FIG. 4. A candidate pure culture microorganism is grown under a wide variety of conditions to maximize the probability that all of its pathways will be expressed. Culture broths are tested for antibiotic activity against a panel of indicator strains for activity against various non-pathogenic microbial strains as well as pathogens, e.g. methicillin-resistant Staphyloccus aureus (MRSA), vancomycin-resistant Enterococcus faecalis (VRE) and strains of fungal pathogens such as Candida albicans that are resistant to azole or polyene drugs. If the crude extract contains one or more bioactive compounds, the extract proceeds to a first CHUMB assessment. Mass spectra, UV spectra, and retention time are collected along with the screening activity data points for each test strain and the activity profiles are stored in the knowledge repository. This knowledge repository allows correlations to be made between pathway class, optimal expression conditions, and antimicrobial spectrum and physical properties. The global analysis of CHUMB assays for a number of growth conditions is referred to as CHUMB-1 analysis. Analysis of CHUMB-1 UV/mass spectral data allows, in some cases, dereplication, and in other cases partial structure elucidation or functional group identification. Based on correlations within the knowledge repository, conditions are selected for scale up fermentation required for structural elucidation. An extraction procedure is used to capture all metabolites from the large-scale fermentations. For example one general procedure described below localizes a given metabolite in one or more of five fractions based on cellular location and polarity. These extracts are also subject to the CHUMB process and then analysed to verify the presence of the metabolites targeted in the CHUMB-1 analysis. Analysis of the general extraction fractions of a given large scale fermentation is referred to as CHUMB-2 analysis.

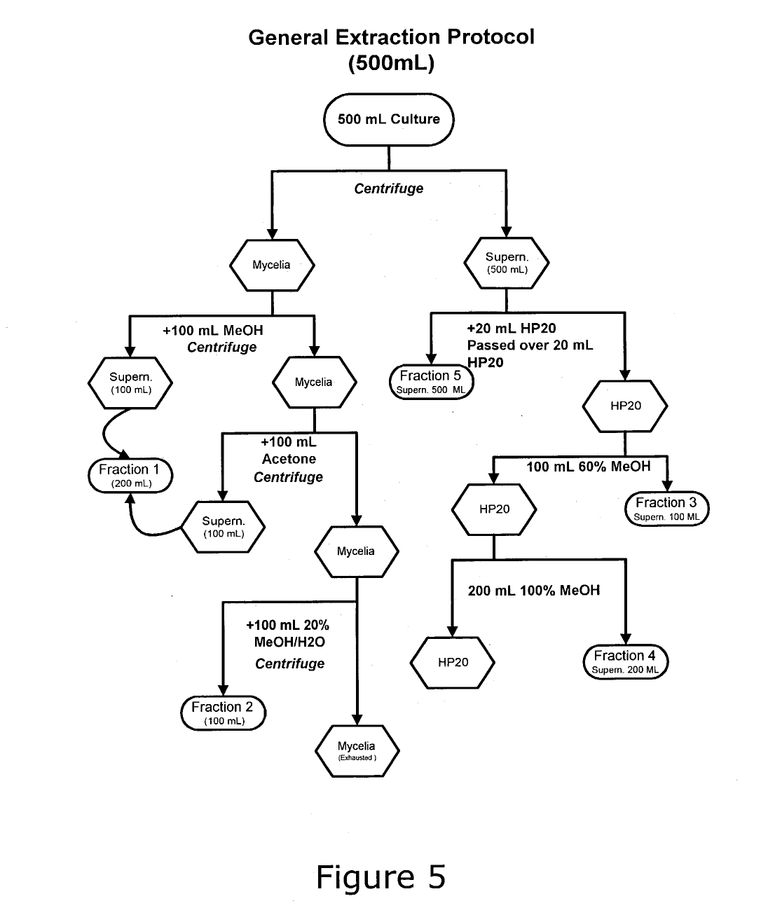

One general extraction procedure, illustrated in FIG. 5 is described as follows. Centrifuge the fermentation broth (500 mI) and decant to separate the supernatant from the mycelia. To the supernatant is added 30 ml of HP-20 resin. This slurry is stirred for 20 minutes after which it is filtered through a short column of HP-20 resin (30 ml). The column is then washed with 100 ml of water. The wash is combined with the initial eluate and labeled as extract no.5. The column is then eluted with 100 ml of 60% MeOH/water and the eluate labeled as extract no.3. The column is then eluted with 100 ml of 100% MeOH and then with 100 ml of acetonitrile. Combine these as extract no.4. To the mycelia is added 100 ml of 100% MeOH, stirred for 10 minutes, centrifuged for 15 minutes, and the supernatant is decanted. To the mycelia is added 100 ml of acetone. The mixture is stirred for 10 minutes, centrifuged for 15 minutes and the supernatant decanted, adding it to the previous methanolic supernatant. This mixture is labelled as extract no.1. To the mycelia is added 100 ml of 20% MeOH/Water. This mixture is stirred for 10 minutes, centrifuged for 15 minutes and decanted. Label this supernatant liquid as extract no.2. Discard spent mycelia.

To summarize, metabolic components for a given organism grown under multiple conditions can be identified by CHUMB-1 analysis and “dereplicated” (distinguished from known compounds) by comparison to a knowledge repository of known compounds, or identified as potentially new compounds. After targets are selected, representing potentially new compounds, scale-up fermentations are performed to produce and isolate sufficient quantities of the compounds for structural elucidation by spectral analysis or other means. The efficiency of the discovery process increases with each chemical structure that is assigned to a biosynthetic pathway in the knowledge repository.

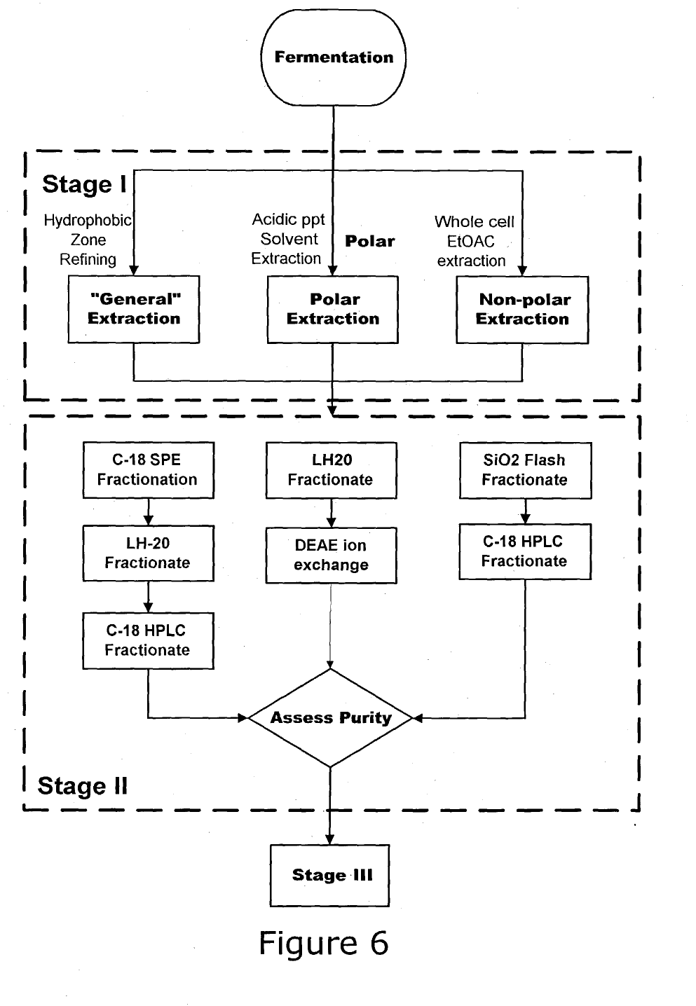

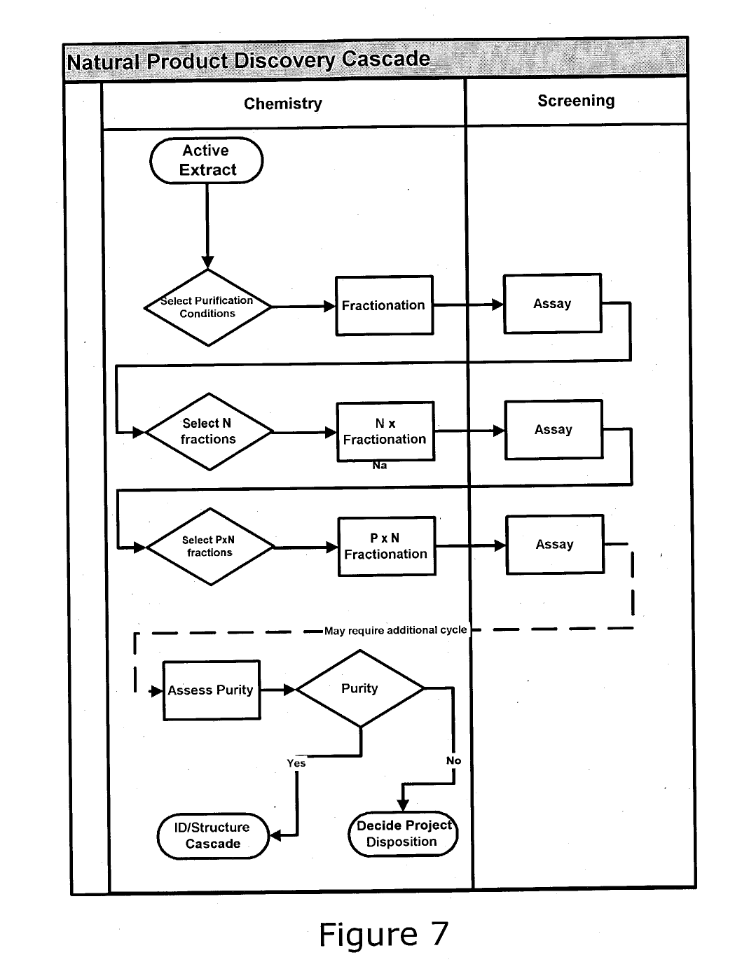

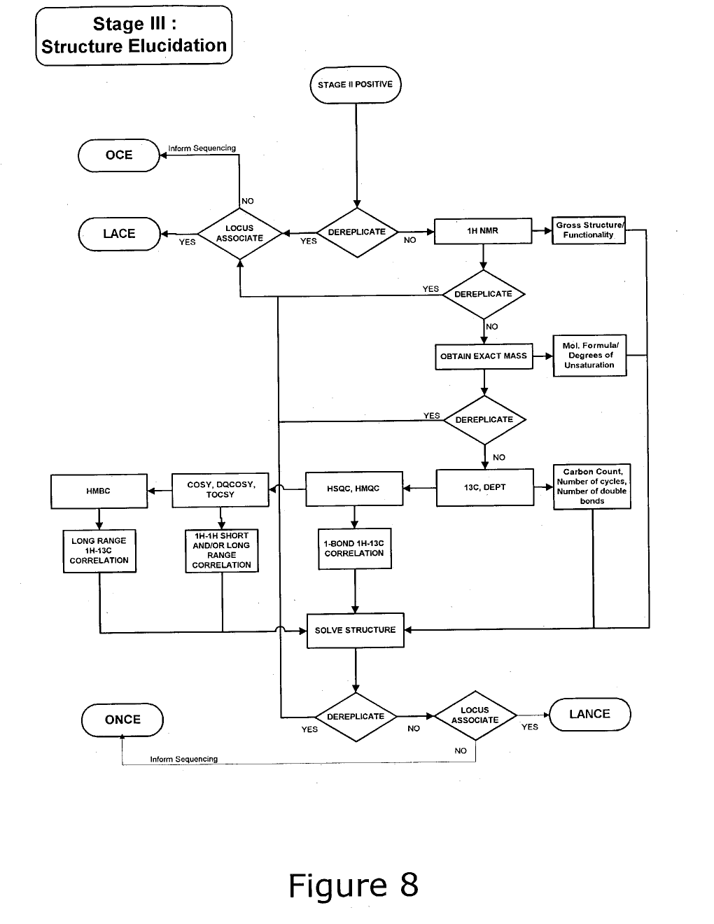

FIGS. 6, 7 and 8 provide an overview of a three-phase genomics-guided extraction/isolation/structure-elucidation protocol that may be used to discover natural product metabolites according to one embodiment of the invention. FIGS. 6, 7 and 8 illustrate a scheme wherein an extract is taken through a three-stage purification process that is designed to rapidly assess if the active component(s) are known compounds or are likely to be new. Genomic information from a knowledge repository facilitates compound identification at each stage by defining the range of chemical compounds that can be expected. Stage I and Stage II (FIGS. 6 and 7) are multi-step purification protocols, and the procedure used depends on whether the target compound is polar or non-polar, for example as may be determined by pre-screening CHUMB and genomics information. Stage II of the protocol is illustrated generally in FIG. 7. Stage III (FIG. 8) provide a structure elucidation cascade. Stage I (FIG. 6) is intended to extract and enrich bioactive components from a fermentation broth. At the end of Stage I there may still be thousands of compounds in the remaining slurry. In one embodiment, Stage I begins with about 500 ml to 2L of crude fermentation broth which, at the end of Stage I extraction and enrichment, is reduced to about 2 ml for use in Stage II (FIG. 7) and Stage III (FIG. 8). The actual steps and order of steps in the extraction process of Stage I may be varied depending on the nature of the target compound. The invention may incorporate standard procedures for isolation of hydrophobic compounds using non-polar solvents such as ethyl acetate or acetone. Other protocols may be adapted or developed to allow for isolation of hydrophilic compounds. Examples of non-polar compounds include polyketides and polysaccharides; examples of polar compounds include peptide-based small molecules such as daptomycin, β-lactams, ramoplanin and vancomycin. In one embodiment, polar compounds are extracted from a fermentation broth by acidic solvent extraction, i.e. if the pH of the slurry is lowered to about pH 3, some polar compounds become soluble in organic solvents. Crude broths are extracted and fractionated using a variety of chromatographic procedures and the initial chemical properties of the active component(s) are determined. Chromatography results may be fed-back to and stored in the knowledge repository and linked to the locus information for the microorganism thereby providing an early opportunity to determine if the active component is a known compound.

One embodiment of the general protocol of FIG. 7 is shown as Stage II in FIG. 6, wherein active components in the remaining slurry produced in Stage I (FIG. 6) may be isolated and identified. The chromatography systems used and order of steps in the purification process may be varied depending on the nature of the target compound. A polar protocol that can be used in the invention involves LH20 fractionation (fractionation by size and polarity), followed by DEAE anionic exchange that fractionates positively charged compounds, and CHUMB. A non-polar protocol that can be used with the invention involves standard silica dioxide fractionation, followed by CHUMB. After purity assessment, the compound continues to stage III, structural elucidation.

FIG. 8 schematically illustrates a Stages III structure elucidation component of a three stage extraction/isolation/structure-elucidation protocol according to one embodiment illustrated in FIGS. 6, 7 and 8. Compounds that are not dereplicatively identified in Stage II (FIG. 6), and thus have the potential or being new chemical entities (NCEs), may be analyzed by UV/visible, infrared, tandem mass spectral and ¹H-NMR, ¹³C-NMR and multidimensional NMR methods to provide definitive structural information. These may include DEPT, HSQC, HMQC, COSY, DQCOSY, TOCSY, and HMBC NMR pulse sequences, which acronyms stand for distortionless enhancement of polarization transfer, heteronuclear single quantum coherence, heteronuclear multiple quantum coherence, correlation spectroscopy, double quantum-filtered correllation spectroscopy, total correlation spectroscopy, and heteronuclear multiple bond coherence respectively. FIG. 8 provides one scheme for structure elucidation. In the embodiment illustrated in FIG. 8, the NMR procedures require an aliquot of the isolate obtained from Stage II (FIG. 6). In the case of peptides, amino acid analysis (PICOTAG or MS/MS analysis) requires just picomole amounts of material. Adequate quantities can be obtained from CHUMB plates to obtain amino acid residue identification. Referring to FIG. 8, the schematic starts with a stage II purified compound having no match among known chemical entities. Further characterization of compounds are conducted and dereplication is again employed to ensure that subsequent steps proceed only when there is no indication that the secondary metabolite of interest corresponds to a known entity. The designation LANCE refers to a locus-associated new chemical entities which means an NCE that is linked to a gene cluster for which there is genomic information; the designation ONCE refers to an orphan new chemical entities which means an NCE that is not yet linked to a gene cluster for which there is genomic information; the designation OCE refers to an orphan chemical entity which means a metabolite that is dereplicated at any point in the structure elucidation cascade, i.e. found to be identical to a previously described compound, and that is not linked to a gene cluster for which there is genomic information; the designation LACE refers to a locus associated chemical entity which means a metabolite that is dereplicated and that is linked to a gene cluster for which there is genomic information.

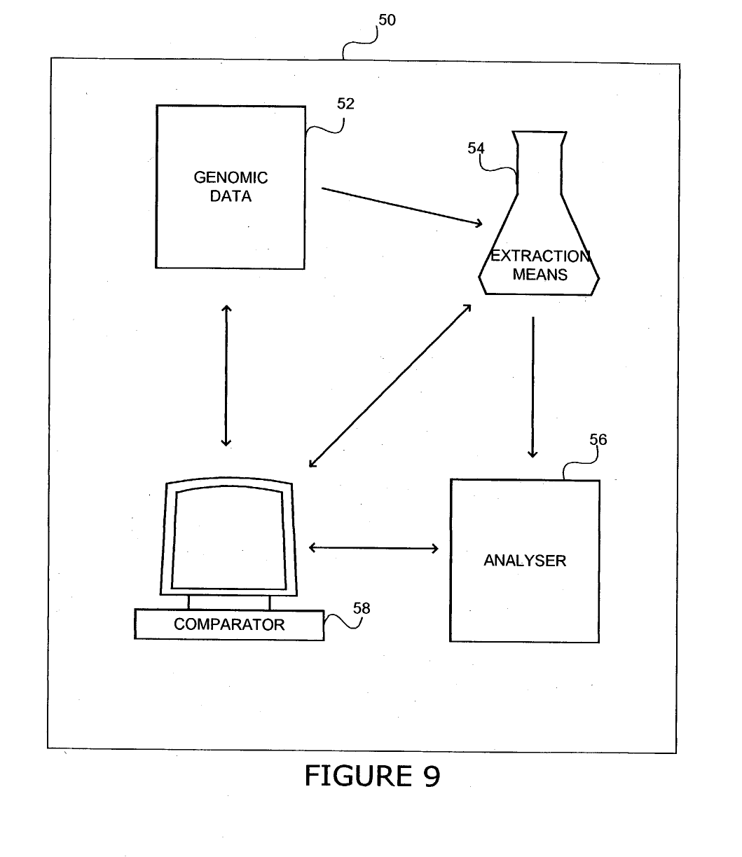

System: The invention provides a system for identifying a secondary metabolite synthesized by a target gene cluster contained within the genome of a microorganism, which system may be computerized or contain a computerized component. FIG. 9 illustrates a system (50) for identifying a secondary metabolite synthesized by a target gene cluster includes genomic data (52), an extraction means (54), an analyser (56) and a comparator (58), each of which is described in more detail below. The genomic data is also referred to as genomic information in the present specification.

An extraction means is used in the system, which is capable of obtaining an extract from the microorganism which contains the metabolite of interest produced by the target gene cluster. Such an extraction means may be a culture system which may incubate the cells under a selected group of conditions, and which thus derives extract from the cells after suitable incubation either by obtaining products exuded by cells in culture, or by disrupting cells at the end of an incubation period. Such methods would be known to or practicable by one skilled in the art.

The system further contains an analyser used to measure chemical, physical or biological properties of metabolites within the extract. As discussed herein, UV spectrum, HPLC, activity assays, chromatography, and other means of detecting chemical, physical or biological properties of metabolites may be used in the analyser component of the system.

The comparator of the system is used to identify, from these measured properties obtained by the analyser, the presence of the metabolite of interest. The comparator may be a computer system adapted to accept inquiries from a user, or may be programmed in such a way as to effect inquiries in a pre-determined manner. The comparator may function not only to effect comparison, but may optionally have interaction with any or all other components of the system, for example by housing data derived from the individual components of the system.

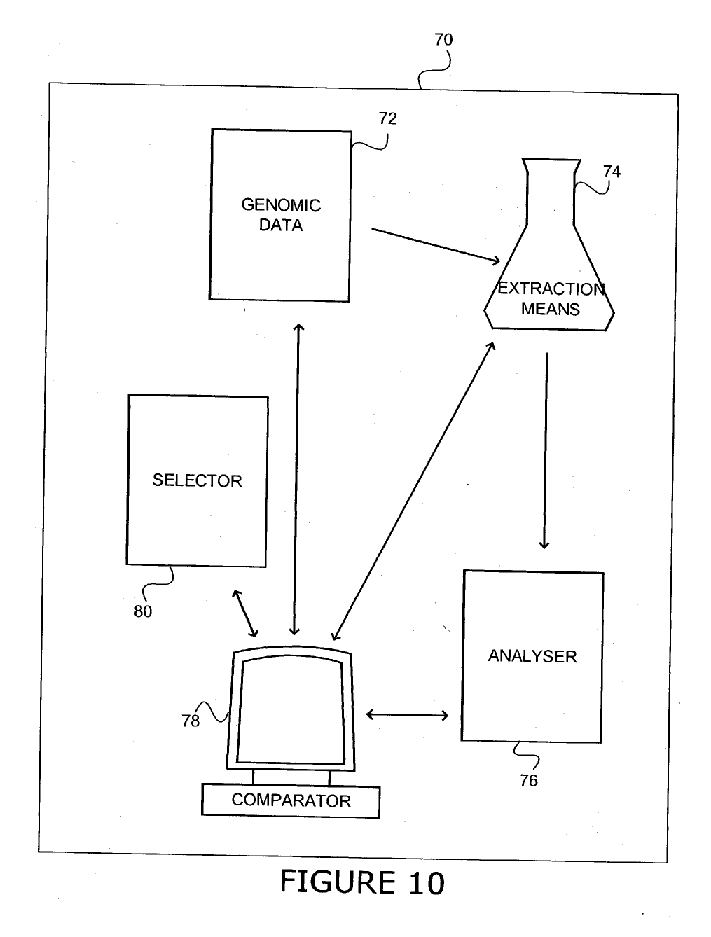

Similarly, the invention provides a system for identifying a secondary metabolite from a pre-selected chemical family. FIG. 10 provides a schematic representation of such a system. The system (70) includes the components discussed above, namely: genomic data (72), an extraction means (74), an analyser (76) and a comparator (78), but also includes a selector (80) for selecting a microorganism containing a target gene cluster. The selector may be, for example, a selectable item accessed from a graphical user interface. In this way, the system according to the invention allows selection of an appropriate microorganism capable of producing a particular desired metabolite from a class (or family) of metabolites on the basis of available genomic data. The comparator may function not only to effect comparison, but may optionally have interaction with any or all other components of the system, for example by housing data derived from the individual components of the system.

Knowledge Repository: According to the invention, a knowledge repository is provided, which houses secondary metabolism data from a microorganism. The repository can be used to identify a secondary metabolite synthesized by a target gene cluster contained within the genome of a microorganism. The repository comprises genomic data confirming the presence of a target gene cluster within a microorganism and genomic information pertaining to the gene cluster. Further, the repository houses extract characterizing data providing chemical, physical or biological properties of metabolites contained in an extract derived from the microorganism. These metabolites include a secondary metabolite attributable to a target gene cluster. Additionally, the repository includes comparative data, representing predicted chemical, physical or biological properties of the secondary metabolite synthesized by the target gene cluster. Within the knowledge repository, the extract-characterizing data is comparable with the comparative data for identifying a secondary metabolite the metabolites in an extract.

A knowledge repository may be, for example, a location at which data is stored or a grouping of data within one or more databases. According to the invention, the knowledge repository allows related information to be stored, added, correlated, compared and retrieved as required. The knowledge repository may be under computer control, and may store a variety of types of information such as chemical, physical and biological properties of a metabolite (for example, structure, molecular mass, UV spectrum or bioactivity), genetic information relating to a microorganism, or culture conditions under which a microorganism produces a metabolite. The knowledge repository may include previously established data obtained through accessing public or private databases, as well as newly generated data obtained according to the invention.

The knowledge repository may provide a “prediction link” between individual records within the repository. For example, genomic data and comparative data (representing expected chemical, physical or biological properties of a metabolite) may be correlated via a prediction link if it is established through actual observation that a metabolite of a target gene cluster possesses the expected properties. Such prediction links formed within the knowledge repository strengthen the predictive value of the knowledge repository when a new microorganism possessing a target gene cluster or a portion thereof is identified. In this way, the knowledge repository advantageously benefits from previously established data and new data added thereto, to predict the potential of a new microorganism (one for which secondary metabolism data has yet to be fully elucidated) to provide a member of a given class or family of compounds.

In related aspects, the invention provides a knowledge repository in which gene cluster information is linked to secondary metabolite production data. The invention further relates to a graphical user interface for accessing the knowledge repository. Further, according to embodiments of the invention, a memory for storing data may be considered a component of the knowledge repository, the memory having a data structure stored therein. The memory may include links between certain types of data. For example, in some embodiments the data representing a chemical structure of a metabolite is linked to a gene cluster or a genetic locus within the genomic data housed in the knowledge repository, thereby increasing the predictive power of the invention and allowing known compounds or compound classes (within a chemical family) to be identified earlier in the purification process.

The invention further provides a memory for storing secondary metabolism data for access by an application program being executed on a data processing system for identifying a secondary metabolite synthesized by a target gene cluster contained within the genome of a microorganism. The memory comprises a data structure stored therein, the data structure including information resident in a database that is used by the application program. This database includes (i) genomic data confirming the presence of a target gene cluster within a microorganism, wherein a putative or confirmed function has been attributed to at least one region of a gene in,.the gene cluster; (ii) extract-characterizing data providing chemical, physical or biological properties of metabolites contained in an extract derived from the microorganism, wherein said metabolites include a secondary metabolite attributable to the target gene cluster; and (iii) comparative data representing expected chemical, physical or biological properties of the secondary metabolite synthesized by the target gene cluster. The extract-characterizing data is comparable with the comparative data for identifying from the metabolites in an extract the secondary metabolite synthesized by the target gene cluster, based on the putative or confirmed function attributed to the at least one region of a gene in a gene cluster.

The invention also relates to a method of building a knowledge repository housing secondary metabolism data from a microorganism. This method comprises the following steps. Genomic data is assembled, confirming the presence of a target gene cluster within a microorganism, wherein a putative or confirmed function has been attributed to at least one region of a gene in the gene cluster. Extract-characterizing data is input, so as to provide chemical, physical or biological properties of metabolites observed in an extract derived from the microorganism, wherein the metabolites include a secondary metabolite attributable to the target gene cluster.

Further, the extract-characterizing data are compared with comparative data representing expected chemical, physical or biological properties of the secondary metabolite synthesized by the target gene cluster. This step allows identification, from the metabolites in an extract, of the secondary metabolite synthesized by the target gene cluster based on the putative or confirmed function attributed to the at least one region of a gene in a gene cluster. Finally, the result of the extract-characterizing step is retained by linking a secondary metabolite identified in the comparing step with the genomic data assembled in the assembling step.

The step of inputting extract-characterizing data may optionally comprise inputting culture conditions under which an extract is derived, and the step of retaining the result may additionally comprise linking culture conditions to both the secondary metabolite identified in the comparing step and the genomic data assembled in the assembling step. The step of inputting extract-characterizing data may comprise inputting a biological property, such as antibacterial, antifungal or anticancer activity.

Similarly, another method of building a knowledge repository housing secondary metabolism data from a microorganism for predicting secondary metabolite production from a target gene cluster based on genomic data is provided according to the invention. This method comprises assembling genomic data confirming the presence of a target gene cluster within a microorganism, wherein a putative or confirmed function has been attributed to at least one region of a gene within the gene cluster. The following steps are also included: extracting a medium containing said microorganism, thereby forming an extract; screening the extract for extract-characterizing data indicative of the presence or absence of a secondary metabolite attributable to the target gene cluster based on a pre-selected chemical, physical or biological property; entering the extract-characterizing data into the knowledge repository; comparing the extract characterizing data with comparative data representing expected chemical, physical or biological properties of a secondary metabolite synthesized by the target gene cluster, so as to identify from the extract a secondary metabolite synthesized by the target gene cluster based on the putative or confirmed function; determining the identity of a secondary metabolite extracted; and affirming within the knowledge repository a correspondence between genomic data, the pre-selected chemical, physical or biological property, and the identity of the secondary metabolite, allowing a cycle of prediction of secondary metabolite production based on genomic data.

Feed Back into Knowledge Repository: The invention contemplates that chemical, physical or biological properties are measured in regard to metabolites produced by microorganisms. Screening activity data-points are collected for each microorganism that enters an expression/screening process. In some embodiments, the activity profiles are stored in a knowledge repository. For example, the results of any bioassay used to determine biological activity are fed-back to and stored in a computer and presented graphically or as a colored bar graph, indicating which of the fractions are bioactive. The activity profiles allow correlations to be made between pathways, chemical class or chemical family, optimal expression conditions and antimicrobial (or other bioactivity) spectrum. Similarly, data regarding physical properties of a metabolite (such as UV spectrum and mass obtained during CHUMB steps) is fed-back and stored in a knowledge repository. This increases the predictive value of the database, as more data is added and more correlations are found, to assist in forming prediction links.

Graphical User Interface: According to the invention, a graphical user interface (GUI) may be provided for subscribing to a knowledge repository. By “subscribing” to the repository, it is meant accessing, adding or modifying data within, producing reports from, or searching within the knowledge repository. The repository houses secondary metabolite data from at least one microorganism for identifying a secondary metabolite synthesized by a target gene cluster. Optionally, data from more than one organism may be housed in the repository, and there is no upper limit on the number of observations or organisms for which data may be housed in the repository. Indeed data derived from thousands of microorganisms may be housed in the repository.

The graphical user interface comprises a genomic access element for accessing from within the knowledge repository genomic data. This genomic data confirms the presence of a target gene cluster within a microorganism, wherein a putative or confirmed function has been attributed to at least one region of a gene in a gene cluster. The genomic access element may be positioned on a computer screen, and may access the genomic data within the repository when a command is received from a user at the interface, for example using a selectable pull-down menu, by entering a microorganism name, or by clicking on (selecting) an icon or other representation of a genomic region of interest.

The graphical user interface also comprises an extract-characterizing access element for accessing from within the knowledge repository chemical, physical or biological properties of metabolites contained in an extract derived from the microorganism. The extract-characterizing access element may be positioned on a computer screen, allowing access to the knowledge repository through a selectable pull-down menu, by entering terms indicative of extract-characterizing properties, or by clicking on (selecting) an icon representing certain extract-characterizing data such as media type, culture conditions, or biological activity. This element may be configured so as to provide searchable access to media composition and growth conditions under which a microorganism extract was obtained. This is a particularly helpful query if a user is attempting to determine conditions under which a certain cryptic pathway is “turned on”, if a metabolite not normally generally produced by a particular organism is shown to be present in a particular extract. Those conditions so located could be used in an effort to turn on similar metabolic pathways in other microorganisms shown to have similar target clusters within their genomic data.

Further, the graphical user interface includes a comparative access element for effecting a comparison of a selected chemical, physical or biological property which may be desired with chemical, physical or biological properties measured or detected within an extract. This comparison is made to allow for identification of a metabolite synthesized by the target gene cluster within a microorganism. Thus, the graphical user interface of the invention allows searchable or query-based access to the knowledge repository of the invention.

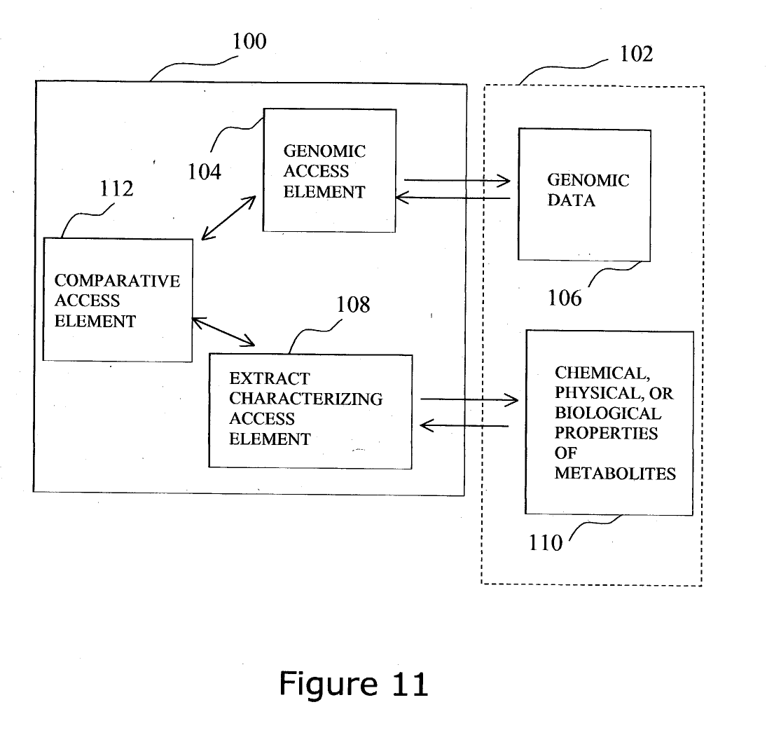

FIG. 11 provides a schematic representation of a typical graphical user interface according to the invention. The graphical user interface (100) is used to subscribe to a knowledge repository (102). The interface comprises a genomic access element (104) for accessing genomic data (106) within the knowledge repository. An extract-characterizing access element (108) is provided for accessing the chemical, physical, or biological properties of metabolites (110) from within the knowledge repository. A comparative access element (112) is also provided which allows a comparison to be effected between an expected or desired property, based on genomic data, with actual properties of metabolites in order to identify a metabolite synthesized by a target gene cluster within a microorganism.

Many variations in the appearance of a graphical user interface (GUI) can be conceived of for organizing and displaying data according to the invention, and these would fall within the scope of the graphical user interface of the invention.

The status of different stages or procedures according to certain embodiments of the invention may be displayed on computer medium in the form of reports illustrated on a computer screen. Such reports may also be produced in printed form. The stages of analysis for each extract may be provided within such a report, and success qualifiers for each stage can be provided.

As an example of such a status report, information relating to the chemistry aspects of a project run using the method or system of the invention can be produced in a “Chemistry Project Report”. The Chemistry Project Report may include such parameters as microbial identification data, extract and medium identification data, the scientist responsible for a particular entry in the report, the date on which an entry was made in the report, or the phase status of a particular extract. The phase status may be, for example, a report of whether a stage of a discovery platform has been completed. Evaluation and monitoring of the phase status may be done in any number of ways, such as by assigning a success qualifier to each discrete state of the natural product discovery cascade. A success qualifier may be, for example, a visual differentiator, such as different colors or patterns displayed on the report to indicate success according to a legend. For example, in a Chemistry Project Report, Stage I processes may involve extraction, initial fractionation, and bioassay of a given microorganism in a media formulation; Stage II processes may involve identifying the active component of the extract and determining its molecular weight via HPLC/MS; and Stage III processes may involve isolation of significant quantities of an active component and its structural elucidation. Each of these stages can be evaluated and the status provided in the report.

If visual differentiators are used, the color of each qualifier can be defined in a legend. As an example of color-based visual differentiators: a green success qualifier can be used to indicate that a project was attempted and the result was positive; a red success qualifier may be used to indicate a project was attempted and negative results were obtained; a yellow success qualifier may be used to indicate that a project was completed; a purple success qualifier can be used to indicate that a project was discontinued; and a blue success qualifier may be used to indicate that a project is ongoing. By using visual differentiators, the Chemistry Project Report produced at the Graphic User Interface provides immediate visual assistance to a user, to a greater extent than is available from simply displaying data values, for example.

The reports available may display any number of columns and/or rows of information, as required, and a comments column may also be used to relate observations on the secondary metabolites and/or activity levels detected in a particular extract.

Other types of reports can be provided, including screening tables representing results for a large scale primary screen of extracts from an organism. Screening results from those organisms within a culture collection may be provided in a report format. In one column of such a report the media growth conditions used can be provided, and various test organisms used to asses biological activity (for example antibacterial or antifungal activity) may be listed in a row so as to provide a biological activity array in table format. Biological activity can be rated according to potency, and groups of organisms with unique activities may be ascertained in this manner and submitted for primary CHUMB analysis.

Once CHUMB analysis is completed, the data may be input into the system so as to build the knowledge repository. This data may be accessed through the graphical user interface. The data may be displayed via a “CHUMB” graph of the CHUMB parameters (C18, HPLC, UV, mass and bioactivity). In a typical CHUMB graph, each point in a chromatogram can be assessed in terms of UV spectrum, mass spectrum, and bioactivity. For example, hundreds of separate CHUMB fractions may be used to construct the graph. This adds a chromatographic dimension to traditional screening data and provides indication of groups of compounds with a broad range of polarities that are active against the various test organisms under various conditions. Investigation of the spectra of the bioactive points is used for identification of known compounds (dereplication) and assignment of possible new chemical entities.

According to the invention, the graphical user interface may be used to illustrate the results of a screening matrix representing extracts derived from any particular organism grown under a variety of conditions. Growth conditions may be displayed on the interface or may be accessed through a hierarchy, the top level of which is displayed on the screening matrix. The matrix may be sortable by clicking on a row header. For example, it is possible for a user to sort by “state”, which displays the activity profile of a given medium across a panel of indicators. This would help group media by similar activity profiles.

The graphical user interface may access sources other than the knowledge repository. For example, the interface may allow the user to access a publicly available or private databases through an internet connection, or based on electronic information stored on a CD. Such databases of known natural products which can be searched by physical properties of a compound include the Dictionary of Natural Products and Antibase. Any appropriate database or website could be accessed by the graphical user interface according to the invention.

The graphical user interface may be used to “dereplicate” a data point for example, if a predicted mass derived from a database of known compounds indicates the presence of a particular metabolite. If the organism of interest was previously shown to make the known compound, the compound can be dereplicated from the information contained in the knowledge repository at this point. For those compounds which are not dereplicated during the CHUMB process, (i.e. have no match in the knowledge repository), such compound can be considered as potential new chemical entities.

The graphical user interface may allow query on the basis of the presence of a particular biosynthetic locus. An identified locus within the knowledge repository may be represented by an icon or other representation that may be selected (clicked on) to allow a user to access information as to what type of metabolites are encoded by this locus.

The graphical user interface may also allow a particular genomic sequence to be “BLASTed” against the genomic information in the database report, which is to say, the sequence (amino acid or nucleic acid) is aligned and compared with other sequences within the knowledge repository for matches as determined using bioinformatics analysis. The sensitivity of such a query (the percentage of identity required to qualify a sequence as a match) may be set by the user.

### EXAMPLE 1

### Discovering and Expressing Cryptic Enediyne Natural Product Biosynthetic Pathways

Genomic information related to a conserved group of genes involved in the synthesis of the highly reactive chromophore ring structure or “warhead” that characterizes all enediynes was generated as described in U.S. Ser. No. 10/152,886 and U.S. Ser. No. 60/398,795. The conserved genes are generally arranged in an operon structure with unidirectional transcription and frequent overlap of translational start and stop codons, suggesting that their gene products are coordinately expressed and functionally related. These genes are from five distinct protein families based on sequence homology and, in some cases, domain organization. The families are referred to as PKSE, TEBC, UNBL, UNBV and UNBU the sequence information for which is provided in U.S. Ser. No. 10/152,886. The PKSE family consists of multimodular polyketide synthases (PKSs) composed of several domains in an unusual order described in more detail below. A putative function was attributed to PKSE, TEBC, UNBL, UNBV and UNBU by comparing their protein sequences to those present in the GenBank nonredundant database. The PKSE family consists of multimodular PKSs composed of several domains in an unusual order. PKSE is distantly related to other types of PKSs. The TEBC proteins were found to be similar to the 4-hydroxybenzoyl-CoA thioesterase (1 BVQ) of Pseudomonas sp. strain CBS-3 in regions of the protein that have been shown to play an important role in catalysis (Benning, M. M. et al., J. Biol. Chem. 273, 33572-33579 (1998)) and thus may be involved in polyketide chain release and/or cyclization. The UNBL, UNBV and UNBU proteins show no significant homology to proteins in the public databases and therefore represent novel protein families that appear to be specific to enediyne biosynthetic loci. PSORT analysis (Nakai, K. & Horton, Trends Biochem. Sci. 24, 34-36 (1999)) of the UNBV proteins predicts that they are secreted proteins having N-terminal signal sequences, while the UNBU proteins are predicted to be integral membrane proteins with seven or eight putative membrane-spanning alpha helices.

The DECIPHER® database (Ecopia BioSciences Inc., St.-Laurent, QC, CANADA) was consulted to identify microorganisms containing the enediyne warhead cassette cluster but not previously reported to produce enediyne compounds. Such cryptic enediyne gene clusters were identified in Amycolatopsis orientalis ATCC 43491 (a known vancomycin producer), Streptomyces ghanaensis NRRL B-1 2104 (a known moenomycin procucer), Kitasatosporia sp. CECT 4991 (a known taxane producer), Micromonospora megalomicea subsp. nigra NRRL 3275 (a known megalomicin producer), Streptomyces cavourensis subsp. washingtonensis NRRL B-8030 (a known chromomycin producer), Saccharothrix aerocolonigenes ATCC 39243 (a known rebeccamycin producer), Streptomyces kaniharaensis ATCC 21070 (a known coformycin producer), Streptomyces citricolor IFO 13005 (a known aristeromycin and neplanocin A producer). The cryptic enediyne biosynthetic loci were identified by the presence of the conserved enediyne warhead cassette genes as well as other flanking genes frequently found in biosynthetic loci encoding other natural product classes.

As PKSE, TEBC, UNBL, UNBV and UNBU are the only genes common to all enediyne loci and the single structural feature found in all known enediynes is the warhead (Nicolaou, K. C. et al., Proc. Nat. Acad. Sci. USA, 90, 5881-5888 (1993)), a genomics-based correlation between PKSE, TEBC, UNBL, UNBV and UNBU genes as a functional unit responsible for the biogenesis of the warhead was established. The PKSEs are likely to generate the carbon skeleton of the warhead by catalysing iterative cycles of acyl-coenzyme A (acyl-CoA) condensation, ketoreduction and dehydration, using an acyl carrier protein (ACP) domain asia covalent attachment site for the growing carbon chain. The PKSEs contain enzymatic domains characteristic of known PKSs, including ketoacyl synthase (KS), acyltransferase (AT), ketoreductase (KR) and dehydratase (DH) domains, as well as ACP domains. Additional analysis of the PKSE sequences further revealed a domain in the C-terminal region of the protein that is similar to 4′-phosphopantetheinyl transferases (PPTases) (Walsh, C. T., etal., Curr. Opin. Chem. Biol. 1, 309-315 (1997)) and is likely to be involved in posttranslational autoactivation of the PKSE. While the functions of the TEBC, UNBL, UNBV and UNBU proteins remain unknown, the strict association of these proteins with the warhead PKS and their presence in all enediyne biosynthetic loci strongly suggests that they play essential roles in the formation, stabilization or transport of the enediyne warhead.

The shared warhead structure provides all enediyne with the ability to damage DNA. The mechanism of action of enediynes involves binding of the enediyne compound to DNA and the warhead chromophore undergoing the thermodynamically favorable Bergman cyclization resulting in strand cleavage of genomic DNA. The biochemical induction assay (BIA) is a modified prophage induction assay that detects agents that damage DNA (Elespuru, R. K. & Yarmolinsky, M. B., Environmental Mutagenesis. 1, 65-78 (1979)). It is predicted that strains harbouring the warhead genes, when cultured in particular fermentation conditions to induce expression of the gene cluster associated with the enediyne genes will produce an enediyne natural product which in turn can be detected using the BIA.

The microorganisms containing the cryptic enediyne biosynthetic loci were grown under multiple culture conditions to obtain extracts containing the enediyne, metabolites. The strains found to contain a putative enediyne biosynthetic locus were cultured in a variety of fermentation media. Organisms were initially grown in 25 ml of TSB seed medium (Kieser, T. et al., Practical Streptomyces Genetics, The John Innes Foundation, Norwich, United Kingdom, (2000)) for 60h at 28° C. and then diluted 30-fold in 25ml production media. Production cultures (25 ml) were incubated for 7 days at 28° C. under constant agitation. Two milliliters of culture were removed and clarified by centrifugation to provide supernatant samples. The rest of the culture (supernatant and mycelia) was extracted with an equal volume of methanol under agitation for 30 min. Extracts were clarified by centrifugation and diluted accordingly in their respective media supplemented with 50% methanol. The BIA was performed as described in Elespuru, R. K. & Yarmolinsky, M. B., Environmental Mutagenesis. 1, 65-78 (1979). Briefly, 10 μl of supernatant or extract and two-fold serial dilutions thereof were applied to agar plates seeded with Escherichia coli BR513 and incubated for 3 hours at 37° C. Soft agar containing 0.7 mg/ml of X-Gal was added onto the plate and colour development was observed within 30 min.

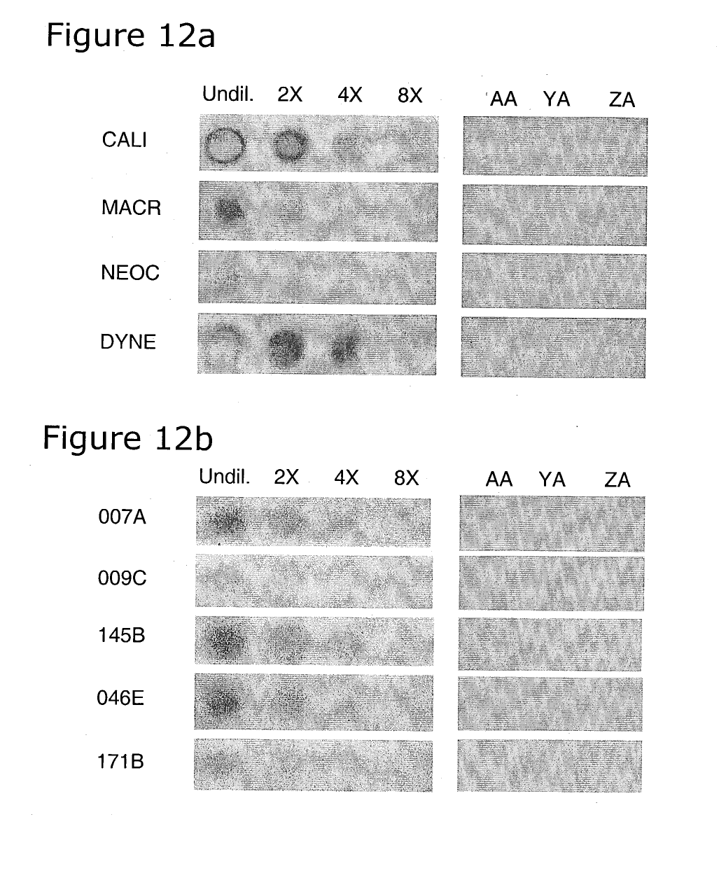

All production media used in this study were assayed alone. Growth of the strains in most media failed to result in detectable BIA activity. However, all strains produced BIA activity when grown in specialized media selected for their ability to support enediyne production (FIG. 12). For calicheamicin, macromomycin and dynemicin, the production media that triggered expression of the enediyne biosynthetic locus were CB, ES and DY. The production media that triggered expression of the neocarzinostatin enediyne biosynthetic locus for was NG. Production media supporting expression of the cryptic enediyne biosynthetic locus in Amycolatopsis orientalis was CB. The production media that supported expression of the cryptic enediyne biosynthetic locus in Streptomyces ghanaensis was KE. The production media that supported expression of the cryptic enediyne biosynthetic locus in Saccharothrix aerocolonigenes was ET. The production media that supported expression of the cryptic enediyne biosynthetic locus in Streptomyces kaniharaensis was ET. The production media that supported expression of the cryptic enediyne biosynthetic locus in Ecopia strain 171 was DY. The production media that supported expression of the cryptic enediyne biosynthetic locus in Streptomyces citricolor was MC. The production media that supported expression of the cryptic enediyne biosynthetic locus in Ecopia strain 046 was MC. The production media that supported expression of the cryptic enediyne biosynthetic locus in Streptomyces cavourensis subsp. washingtonensis was SP. Examples of media not supporting enediyne production include CECT media 32 and 131 (Colección Espa\ddot{n}ola de Cultivos Tipo, Valencia, Spain) herein referred to as media YA and ZA, respectively.

The data generated, including (i) the presence of the PKSE, TEBC, UNBL, UNBU and UNBV genes in each of the microorganisms, notably those not previously reported to produce an enediyne metabolite; (ii) the putative function attributed to the PKSE, TEBC, UNBL, UNBU and UNBV proteins in the enediyne loci; (iii) the multiple culture conditions under which the strains were grown; and (iv) the results of the biochemical induction assay and other bioassays were added to the DECIPHER® database. These data facilitates subsequent comparisons and dereplication of enediyne activities.

### EXAMPLE 2

### Isolation and Structure Elucidation of a Metabolite From a Cryptic Biosynthetic Locus

The systems, methods and knowledge repository of the invention can be used to isolate and elucidate the structure of a metabolite synthesized by a cryptic biosynthetic locus, the product of which is unknown. A sample of the organism Streptomyces cattleya (NRRL 8057) was obtained from the Agricultural Research Service Culture Collection, Peoria, Ill. 61604). A literature search (PubMed) revealed Streptomyces cattleya (NRRL 8057) had not been reported to produce any natural products other than thienamycin and other beta-lactam class compounds (U.S. Pat. No. 3,950,357).

Streptomyces cattleya was subject to the genome scanning method described in U.S. Ser. No. 10/232,370 which resulted in the discovery in the Streptomyces cattleya genome of at least 12 putative natural product biosynthetic loci. These were further characterized by sequence analysis and determined to be distinct biosynthetic loci. Sequence analysis was performed using a 3700 ABI capillary electrophoresis DNA sequencer (Applied Biosystems) and open reading frames were identified from the sequence information. The DNA sequences of the ORFs were translated into amino acid sequences and compared to the National Center for Biotechnology Information (NCBI) nonredundant protein database using the BLASTP algorithm with the default parameters (Altschul et al., supra). Sequence similarity with known proteins of defined function resulted in a putative function being attributed to a number of genes in each of the 12 biosynthetic loci. Of the 12 biosynthetic loci discovered six of them included putative polyketide synthases (PKS) of different varieties based on domain organization.

Streptomyces cattleya was grown in six media formulations, namely BA, DA, EA, KA, NA, OA, for a period of 7 days. Non-polar extraction procedures were employed to capture polyketide based natural products from the-culture broths. An equal volume of ethyl acetate was added to the whole broth, which was subsequently agitated on an orbital shaker for 30 minutes. The organic layer was separated,.dried over magnesium sulfate, and evaporated to yield a crude extract. The extracts were analyzed by thin-layer chromatography and overlay bioassay using several indicator strains (B. subtillis, S. aureus, E. coli, C. albicans, M. luteus, K. pneumonia, P. aeruginosa). Multiple zones of antimicrobial activity were observed in the overlay assays in the extracts derived from the various media. These antimicrobial/antifungal activities are commonly associated with secondary metabolites in Steptomyces and provide convenient assays which can be used to follow progress in purification (bioassay-guided fractionation). Extracts from media DA exhibited substantial Micrococcus luteus activity, and was selected for purification by flash chromatography (SiO₂ plug, 5% MeOH/CH₂Cl₂-100% MeOH) followed by Sephadex LH-20 chromatography (100% MeOH) resulting in a compound that was pure by TLC analysis. ¹H NMR analysis verified that the compound was substantially pure and suggested a polyketide class molecule with multiple double bonds, as evidenced by peaks at 5.5-6.5 ppm (consistent with alkenic double bonds), peaks at 3.5-4.5 (consistent with hydroxyl attached C—H bonds), and 0.5-3 (consistent with alkyl groups).

Genomics information from a knowledge repository assisted in the structure elucidation process. The DECIPHER® database was consulted to associate the measured chemical, physical and biological properties of the polyketide metabolite with one of the “cryptic” biosynthetic loci (the target locus) from Streptomyces cattleya. PKS domain identification was performed on the target locus. Genomics analysis allowed deduction of a biosynthetic scheme for production of the pqlyketide metabolite by the target locus, using bioinformatic analysis of the polyketide chain and comparative analysis with the structure of other PKS enzymes in the DECIPHER® database. In particular, the analysis suggested domain strings from which various structural elements were derived. A portion of the genomic deductions and the corresponding structural deductions are represented below:

[KS-IX-KR-MT-ACP][KS-IX-KR-ACP][KS-IX-ACP]
[C-A(Gly_)-ACP][KS][IX-DH-KR-ACP][KS-IX-DH-KR-MT-ACP][KS-IX-ACP][KS-IX-KR-ACP]
[KS-IX-KR-AOP][KS][DH-ACP-KR][KS-IX-DH-KR-ACP][KS-IX-DH-KR-ACP]

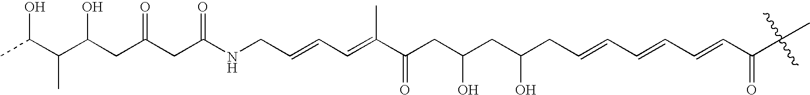

where abbreviations describe processive enzymatic activities or other functions corresponding to ketoacyl synthase (KS), acyltransferase interaction domain (IX), ketoreductase (KR), dehydratase (DH), and enoyl reductase (ER), acyl carrier protein (ACP), methyltrasnferase (MT), and thioesterase (TE) activity involvged in polyketide synthesis, as well as condensation (C) and adenylation (A) activities.

These structural elements were used as possible starting points for structure elucidation studies with multidimensional NMR experiments such as DQCOSY, TOCSY, HSQC, and HMBC. The structural elements deduced from the genomic information matched the experimental NMR data and facilitated the solving of partial structures. The partial structures thus obtained were used to query a database of known natural products and the known compound L-681,217 was identified. The reported spectroscopic data for compound L-681,217 was an exact match to the spectroscopic data collected for the compound isolated from Streptomyces cattleya. The structure of compound L-681,217 is shown below.

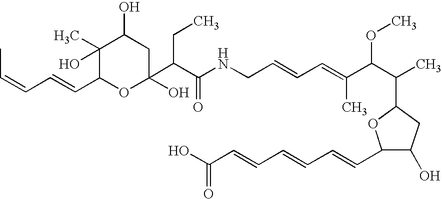

The structure of compound L-681,217 was associated with the biosynthetic locus from Streptomyces cattleya and a link between the structure data and genomics data was made in the DECIPHER® database. This association was, in turn, used to link or associate a separate locus in another organism with a structurally similar compound that is known to be produced by that organism (Streptomyces filippiniensis, heneicomycin). In particular, a comparison of the structures of L-681,217 and heneicomycin led to the prediction that a domain string would be found in the heneicomycin-producer Streptomyces filippiniensis. In support of this prediction, a target locus encoding such a domain string was identified in the genomic data from Streptomyces filippiniensis, as shown below:

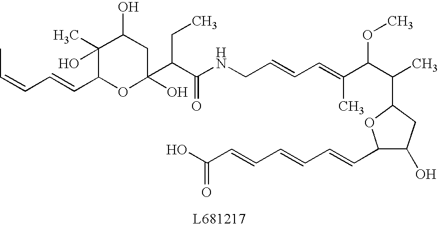

Domains of L681217 locus

[TP]

[ACP][KS-IX-ACP][KS]

[DH-ACP-KR][KS-IX-KR-MT-ACP][KS-IX-KR-ACP][KS-IX-ACP]

[C-A(Gly_)-ACP][KS]

[IX-DH-KR-ACP][KS-IX-DH-KR-MT-ACP][KS-IX-ACP][KS-IX-KR-ACP][KS-IX-KR-ACP][KS]

[DH-ACP-KR][KS-IX-DH-KR-ACP]

[KS-IX-DH-KR-ACP][ks-at]

[AT][AT][NPDC-XX]

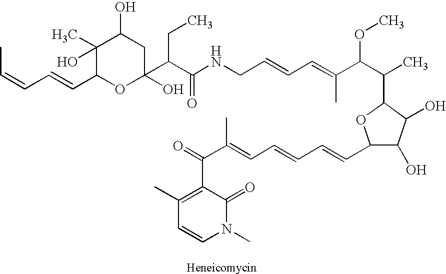

Partial domain string

. . . [ACP][KS-IX-KR-ACP][KS]

[DH-ACP-KR][KS-IX-KR-MT-ACP][KS-IX-KR-ACP][KS-IX-ACP]

[C-A(Gly_)-ACP][KS]

[DH-KR-ACP][KS-IX-DH-KR-MT][KS-IX-ACP][KS-IX-KR-ACP]KS-IX-ACP][KS]

### EXAMPLE 3

### Identifying a Secondary Metabolite of a Pre-selected Chemical Family

The methods, systems and knowledge repositories of the invention can be used to identify a secondary metabolite of a pre-selected chemical family. In this example we describe the identification of the antifungal polyketide Ayfactin, a member of the pre-selected chemical family of “polyenes”.

A knowledge repository was consulted to determine chemical family data for a polyene polyketide. A target gene cluster encoding a putative polyene metabolite was identified based on bioinformatic analysis of genomic information present in the DECIPHER® database (Ecopia Biosciences Inc., St.-Laurent, Canada). The target gene cluster encodes polyketide synthases as well as other proteins similar to those encoded by previously sequenced antifungal polyene biosynthetic loci such as those for partricin, candicidin and nystatin. In particular, the domain structure of the sequenced polyketide synthases includes a partial domain string deduced to be . . . DH-KR-ACP][KS-AT-DH-KR-ACP][KS-AT-DH-KR-ACP][KS-AT-DH-KR-ACP][KS-AT-DH-KR-ACP][KS-AT-DH-KR-ACP][KS-AT-DH-KR-ACP]. . . . corresponding to the synthesis of a polyketide chain with seven or more conjugated double bonds, a structural feature consistent with polyenes such as candicidin. All the AT domains in the domain string were predicted to be specific for malonyl-CoA extender units. The gene cluster also includes genes that are most closely related to genes found in the Streptomyces griseus IMRU 3570 biosynthetic gene cluster encoding candicidin, a polyene compound. These genes include a para-aminobenzoic acid synthase that displays 77% identity and 82% similarity to a synthase in the candicidin cluster (GenBank accession CAC22117); a thioesterase that displays 69% identity and 81% similarity to a thioesterase in the candicidin cluster (GenBank accession CAC22116); and an aminotransferase that displays 79% identity and 89% similarity to an aminotransferase in the candicidin cluster (GenBank accession CAC22113).

The microorganism containing the target gene cluster identified from the DECIPHER® database (designated herein as organism 100) was one from the Ecopia culture collection. Organism 100 had been analyzed using the genome scanning method referred to in Example 1 which resulted in the discovery of several natural product biosynthetic loci, seven of which were further characterized by high-throughput sequencing. The results of the genome scanning and the high throughput sequencing had been entered into the DECIPHER® database. Thus, organism 100 was predicted to contain a biosynthetic locus (designated. herein as locus 100C) coding for the production of a putative antifungal polyene containing seven or more conjugated double bonds.

An extract containing the putative polyene was obtained from organism 100 using a metabolomic approach to identify conditions under which the product of locus 100C was expressed. This approach obtains analytical measurement of all low molecular weight metabolites in a given organism at a specific time when grown under specific culture conditions. Organism 100 was grown in 48 different media, namely AA, AB, AC, BA, CA, CB, CI, DA, DY, DZ, EA, ES, ET, FA, GA, IB, JA, KA, KE, LA, MA, MC, MU, NA, NE, NF, NG, OA, PA, PB, QB, RA, RB, RC, RM, SF, SP, TA, VA, VB, WA, WS, XA, YA, ZA. Metabolites were extracted from whole cell cultures by adding of an equal volume of methanol. After removal of solid debris, the extract was concentrated and injected into an HPLC/MS system in which the metabolites were analyzed to obtain UV and mass data and purified fractions are collected in 96-well plates and assayed for multiple activities including antibiotic activity against gram-positive and gram-negative bacteria, and fungi. Analysis of the chromatographic and bioactivity profiles indicated the presence of a potent antifungal activity in a number of extracts. For example, media RM produced substantial quantities of a chromatographically distinct compound that discplayed antifungal activity against Candida indicators.

Finally, the extracts generated by growth of organism 100 under each of the 48 media were analyzed for metabolites having physical, chemical and biological characteristics of polyenes. This analysis identified a compound of mass 1113 Da having an extended UV chromophore consistent with a heptaene (i.e. having 7 conjugated double bonds) and antifungal activity. Searching a database of greater than 25000 known microbial natural products with this mass, UV, and bioactivity data provided conclusive evidence that the polyene is the known antifungal agent ayfactin, the structure of which is shown below.

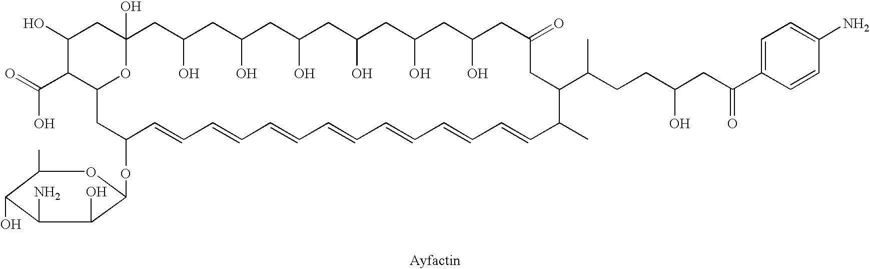

The measured chemical, physical and biological properties of the product of locus 100C were found to be consistent with the reported chemical, physical and biological properties for ayfactin, and are in precise agreement with the bioinformatic predictions made, in regard to an antifungal polyene. The DECIPHER® database was updated to establish a link that associates locus 100C in organism 100 with the chemical structure of ayfactin.

### EXAMPLE 4

### Detection of a Lipopeptide Metabolite From Streptomyces refuineus Subsp. thermotolerans NRRL 3143

Lipopeptides are natural products that exhibit potent, broad-spectrum antibiotic activity with a high potential for biotechnological and pharmaceutical applications as antimicrobial, antifungal, or antiviral agents. A single microorganism may produce a mixture of related lipopeptides that differ in the lipid moiety that is attached to the peptide core via a free amine, usually the N-terminal amine of the peptide core. The lipid moiety can have a major influence on the biological properties of lipopeptide natural products.

Lipopeptides produced by bacteria are synthesized nonribosomally on large multifunctional proteins termed nonribosomal peptide synthetases (NRPSs) (Doekel and Marahiel, 2001, Metabolic Engineering, Vol. 3, pp. 64-77). NRPSs are modular proteins that consist of one or more polyfunctional polypeptides each of which is made up of modules. The amino-terminal to carboxy-terminal order and specificities of the individual modules correspond to the sequential order and identity of the amino acid residues of the peptide product. Each NRPS module recognizes a specific amino acid substrate and catalyzes the stepwise condensation to form a growing peptide chain. The identity of the amino acid recognized by a particular unit can be determined by comparison with other units of known specificity (Challis and Ravel, 2000, FEMS Microbiology Letters, Vol. 187, pp. 111-114). In many peptide synthetases, there is a strict correlation between the order of repeated units in a peptide synthetase and the order in which the respective amino acids appear in the peptide product, making it possible to correlate peptides of known structure with putative genes encoding their synthesis, as demonstrated by the identification of the mycobactin biosynthetic gene cluster from the genome of Mycobacterium tuberculosis (Quadri et al., 1998, Chem. Biol. Vol. 5, pp. 631-645).

The modules of a peptide synthetase are composed of smaller units or “domains” that each carry out a specific role in the recognition, activation, modification and joining of amino acid precursors to form the peptide product. One type of domain, the adenylation (A) domain, is responsible for selectively recognizing and activating the amino acid that is to be incorporated by a particular unit of the peptide synthetase. The activated amino acid is covalently attached to the peptide synthetase through another type of domain, the thiolation (T) domain, that is generally located adjacent to the A domain. Amino acids joined to successive units of the peptide synthetase are subsequently covalently linked together by the formation of amide bonds catalyzed by another type of domain, the condensation (C) domain. NRPS modules can also occasionally contain additional functional domains that carry out auxiliary reactions, the most common being epimerization of an amino acid substrate from the L- to the D-form. This reaction is catalyzed by a domain referred to as an epimerization (E) domain that is generally located adjacent to the T domain of a given NRPS module. Thus, a typical NRPS module has the following domain organization: C-A-T-(E).

Lipopeptides differ from regular peptides in that they contain a lipid moiety usually attached at the N-terminal amine of the peptide core structure. In contrast to regular peptides, in lipopeptide-encoding NRPS clusters the adenylation domain responsible for the activation and tethering of the first amino acid residue of the peptide core is preceded by an unusual condensation domain (C-domain). The genomic information pertaining to the unusual C-domain was generated as described in co-pending applications U.S. Ser. No. 10/329,027 filed Dec. 24, 2002 entitled Compositions, methods and systems for discovery of lipopeptides and U.S. Ser. No. XX/XXX,XXX also filed on Dec. 24, 2002 and entitled Genes and proteins involved in the biosynthesis of lipopeptides, the contents of which are incorporated herein by reference. The unusual C-domain is referred to as an “acyl-specific C-domain” in co-pending applications U.S. Ser. No. 10/329,027 and XX/XXX,XXX. The presence of an acyl-specific C-domain in an NRPS system along with the specific location of this domain in the starter module of the NRPS system indicate that the product encoded by the NRPS system is likely to be a lipopeptide.

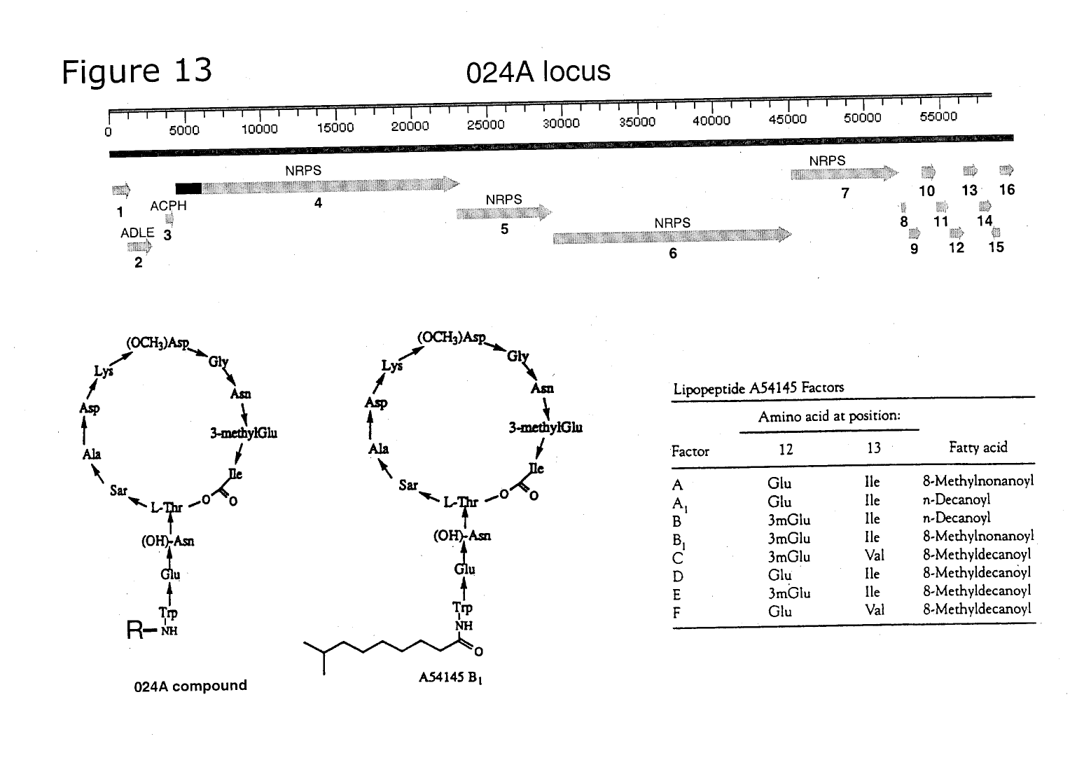

To search for microorganisms that may produce lipopeptides, the DECIPHER® database was consulted to identify microorganisms which contain in their genome an acyl-specific C-domain. One of the microorganisms selected from the DECIPHER® database that clearly contained an acyl-specific C-domain was Streptomyces refuineus NRRL 3143. Further analysis, described in detail in co-pending applications U.S. Ser. No. 10/329,027 and U.S. Ser. No. XX/XXX,XXX, established that this unusual condensation domain was contained in a large NRPS system in Streptomyces refuineus, herein referred to as locus 024A. The precise location of the acyl-specific C-domain was determined to be in the starter loading domain of the NRPS system, indicating that 024A was encoding an N-acylated lipopeptide product (FIG. 13)

Analysis of genomic information contained in the DECIPHER® database allowed the prediction that the NRPS system containing the unusual C-domain in the Streptomyces refuineus 024A locus would direct the synthesis of a polypeptide scaffold identical to that of the known lipopeptide A54145 produced by Streptomyces fradiae (FIG. 13). The genetic locus responsible for biosynthesis of the lipopeptide A54145, herein referred to as A541, is present in the DECIPHER® database. The overall genetic similarity observed between the 024A and A541 biosynthetic loci also indicated that both loci would be expressed under similar growth conditions in the two Streptomyces species (U.S. Ser. No. XX/XXX,XXX and Zazopoulos et al., 2003, Nature Biotechnol., Vol 21) Based on the prediction of structural similarity between the two compounds, it was also expected that the 024A-encoded lipopeptide would have chemical, physical and biological properties similar to those of A54145.

A patent database was then consulted to identify culture conditions under which lipopeptide A54145 in Streptomyces fradiae is expressed (U.S. Pat. No. 4,977,083). Streptomyces fradiae and Streptomyces refuineus were grown under identical culture conditions to assess induction of locus 024A and determine the nature of the specified product.

Both microorganisms were grown at 30° C. for 48 hour in a rotary shaker in 25 mL of a seed medium consisting of glucose (10 g/L), potato starch (30 g/L), soy flour (20 g/L), Pharmamedia (20 g/L), and CaCO₃ (2 g/L) in tap water. Five mL of this seed culture was used to inoculate 500 mL of production media in a 4L baffled flask. Production media consisted of glucose (25 g/L), soy grits (18.75 g/L), Blackstrap molasses (3.75 g/L), casein (1.25 g/L), sodium acetate (8 g/L), and CaCO₃ (3.13 g/L) in tap water, and proceeded for 7 days at 30° C. on a rotary shaker. The production culture was centrifuged and filtered to remove mycelia and solid matter. The pH was adjusted to 6.4 and 46 mL of Diaion HP20 was added and stirred for 30 minutes. HP20 resin was collected by Buchner filtration and washed successively with 140 mL water and 90 mL 15% CH₃CN/H₂O, and the wash was discarded. HP20 resin was thenleluted with 140 mL 50% CH₃CN/H₂O (fraction HP20 E2). This pool was passed over a 5 mL Amberlite IRA67 column. (acetate cycle) and the flow through (fraction IRA FT) was reserved for bioassay. The column was washed with 25 mL 50% CH₃CN/H₂O and eluted with 25 mL 50% CH₃CN/H₂O containing 0.1 N HOAc (fraction IRA E1), and then eluted with 25 mL 50% CH₃CN/H₂O containing 1.0 N HOAC (fraction IRA E2). Biological activity was followed during purification by bioassay with Micrococcus luteus in Nutrient Agar containing 5 mM CaCl₂.

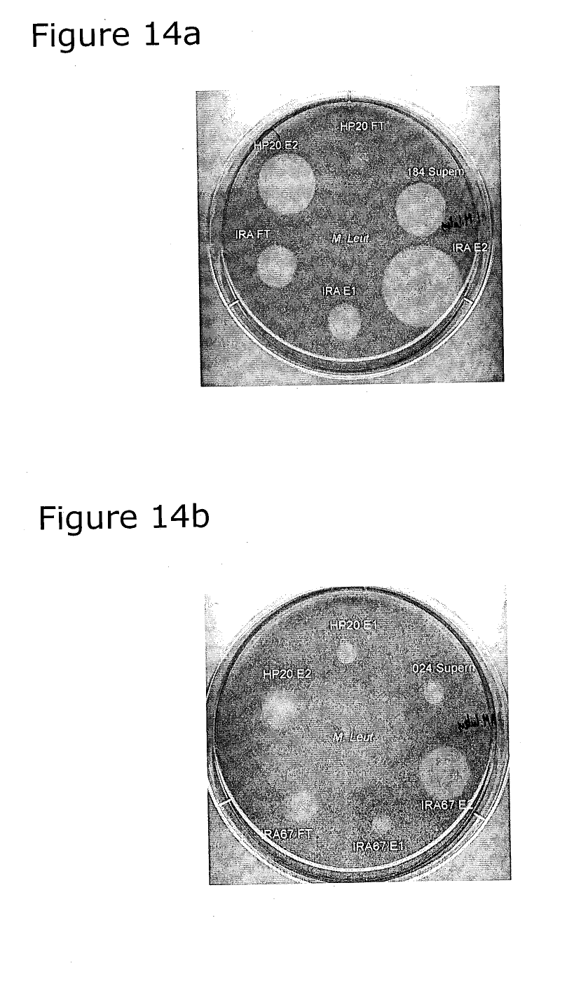

FIG. 14a is a photograph of a plate generated during extraction of an anionic lipopeptide from Streptomyces fradiae, showing an enrichment of activity based on IRA67 anion exchange chromatography consistent with expression of an acidic lipopeptide. This activity is concentrated during the extraction procedure as indicated by the increased diameter of lysis rings. A54145 was detected via HPLC/MS in fraction IRA E2 as evidenced by mass ion ES²⁺=830.5 consistent with the structures of A54145C,D (U.S. Pat. No. 4,994,270). FIG. 14b is a photograph of a plate generated during a similar extraction scheme performed on extracts from Streptomyces refuineus NRRL 3143, showing a similar enrichment of activity based on IRA67 anion exchange chromatography consistent with expression of an acidic lipopeptide. This activity is concentrated during the extraction procedure as indicated by the increased diameter of lysis rings. A mass ion of ES²⁺=830.5, identical to that of A54145, was present in fraction IRA E2 confirming that an N-acylated acidic lipopeptide, identical to A54145C and D, is produced by 024A in Streptomyces refuineus subsp. thermotolerans as predicted from the genomic data contained in the DECIPHER® database.

### EXAMPLE 5

### Identifying a Novel Polyketide From Cryptic Biosynthetic loci via Metabolomic Analysis

Streptomyces aizunensis was subject to the genome scanning method described in Example 1, which resulted in the discovery in the Streptomyces aizunensis genome of many putative natural product biosynthetic loci, five of which were further characterized by sequence analysis and determined to be distinct biosynthetic loci. Of the five biosynthetic loci analyzed, three contained NRPS genes and were predicted to encode for the production of peptides (locus designations 023B, 023C, and 023F), and one was predicted to code for the production of a large polyketide (locus designation 023D). Based upon the genomic information approximate chemical structures were predicted for compounds encoded by loci 023B, 023C, 023F and 023D.

| | | | | | | |
|---|---|---|---|---|---|---|
| | Mass | | | | | |
| Locus | Range | UV | Poss Activity | Class | AA Composition | Notes |

| | | | | | | |
|---|---|---|---|---|---|---|
| 023B | >300 | none | — | Glycosylated | Ile/Leu dimer | |
| | | pred. | | dipeptide | | |
| 023C | >2000 | 250, 280 | antibacterial | glycosylated | XNXGNXFGXXXX | multiple |
| | | | | lipopetide | NNNDDXNAGXA | glycosyl |
| | | | | | ADX | transferases |
| 023D | >1199 | >300 | antifungal | polyketide | n/a | 26 modules, |
| | | | | | | multiple double |
| | | | | | | bonds, |
| | | | | | | glycosyl |
| | | | | | | transferase |
| | | | | | | and deoxy |
| | | | | | | sugar genes. |
| 023F | >1000 | 280 | | decapeptide | XXVXXXXXXN | |
| SRCB | >300 | none | Broad | streptothicin | | |
| | | pred. | spectrum | | | |

A metabolomics approach was subsequently used to identify conditions under which to express secondary metabolites, analyze them, and correlate them to the above biosynthetic loci. This approach obtains analytical measurement of all low molecular weight metabolites (0-5000 Da) in a given organism at a specific time under specific culture conditions. Streptomyces aizunensis was grown in 48 different media, namely AA, AB, AC, BA, CA, CB, CI, DA, DY, DZ, EA, ES, ET, FA, GA, IB, JA, KA, KE, LA, MA, MC, MU, NA, NE, NF, NG, OA, PA, PB, QB, RA, RB, RC, RM, SF, SP, TA, VA, VB, WA, WS, XA, YA, ZA, many of which are representative of media reported to support the production of a wide range of natural products. Metabolites were extracted from whole cell cultures by adding an equal volume of methanol. After removal of solid debris, the extracts were concentrated and analyzed by the CHUMB method. Analysis of the chromatographic and bioactivity profiles indicated the presence, in a number of extracts, of a chromatographically distinct peak with a molecular ion of 1297 Da (1296.1 ES-), and a fragment of 1131 Da (ES-) and UV maxima of 317.77, 332.77 and 350.77. For example, growth in medium QB resulted in the production of substantial quantities of this chromatographically distinct compound, hereafter referred to as ECO-02301. ECO-02301 demonstrated antibacterial activity against Staphylococcus aureus and enterococci, as well as antifungal activity against several Candida species. The physical and biological data for ECO-02301 suggested a large natural product with multiple conjugated double bonds. Inspecting the biosynthetic loci for Streptomyces aizunensis sidentified locus 023D as a likely candidate. This locus contained approximately 26 modules of polyketide synthase, consistent with the observed mass of ECO-02301, as well as a glycosyl transferase, deoxyhexose biosynthetic genes and auxiliary genes of unknown function. The mass fragment of 1131.9 Da was consistent with the loss of a deoxyhexose moiety (deoxyhexose mass=164.16) further supporting the hypothesis that locus 023D directs the production of ECO-02301. The predicted domain sequence of locus 023 D was:

[ACP][KS-AT(M)-KR-ACP][KS-AT(M)-KR-ACP][KS-AT(M)-
DH-KR-ACP][KS-AT(M)-KR-ACP][KS-AT(M)-KR-ACP][KS-
AT(M)-DH^{‡}-KR-ACP][KS-AT(M)-KR-ACP][KS-AT(M)-DH-KR-
ACP][KS-AT(M)-KR-ACP][KS-AT(M)-KR-ACP][KS-AT(M)-
DH^{‡}-KR-ACP][KS-AT(MM)-KR*-ACP][KS-AT(M)-KR-ACP]
[KS-AT(M)-DH-KR-ACP][KS-AT(M)-DH-KR-ACP][KS-AT(M)-
DH-KR-ACP][KS-AT(M)-DH-KR-ACP][KS-AT(M)-DH-KR-ACP]
[KS-AT(MM)-KR-ACP][KS-AT(MM)-KR-ACP][KS-AT(M)-DH^{‡}-
KR-ACP][KS-AT(M)-DH-ER-KR-ACP][KS-AT(M)-DH-KR-ACP]
[KS-AT(M)-DH-KR-ACP][KS-AT(M)-DH-KR-ACP][KS-AT(M)-
DH-KR-ACP-TE

where abbreviations descibe processive enzymatic activities corresponding to ketoacyl synthase (KS), acyltransferase (AT), ketoreductase (KR), dehydratase (DH), and enoyl reductase (ER) activity, as well as acyl carrier protein (ACP) and thioesterase (TE) activity. The specificities of AT domains are also indicated (m, malonyl; mm, methyl malonyl). Asterisk (*) indicates a domain that was predicted to be inactive and ‡ indicates domains whose activity could not be determined based on sequence deduction.

Streptomyces aizunensis was then grown in medium QB in a larger scale fermentations (0.5 L) for seven days and extracted by stirring the pelleted mycelia with an equal volume of methanol, followed by clarification by centrifugation. The extract was then adsorbed onto Diaion HP-20 resin via rotary evaporation onto HP-20 beads and eluted with a methanol step gradient. Fractions containing ECO-02301 were pooled and chromatographed via preparative HPLC chromatography (C-18 ODS) to produce pure ECO-02301. Using the PKS-deduced structure of locus 023D as a structural template accelerated the structural elucidation by NMR spectroscopy, which revealed the structure of ECO-02301 to be a large glycosylated linear polyeneic compound with an unusual amidohydroxycyclopentenone moiety as shown below.

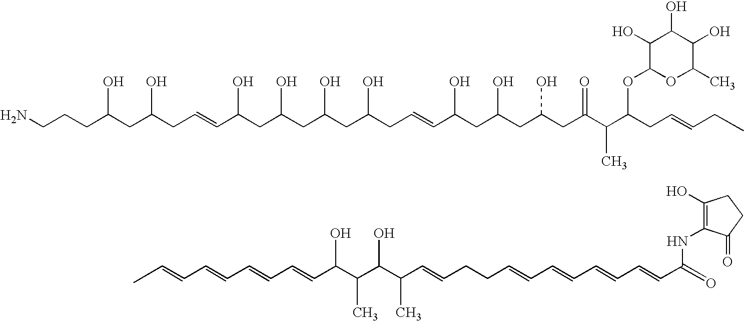

A search of the extant chemical literature and chemical databases revealed that this compound was not previously described and is thus a new chemical entity (NCE). The polyketide backbone and sugar portion of ECO-02301 correlated well with the deduced chemical structure of biosynthetic locus 023D. The polyketide backbone of ECO-02301 is similar to the compound linearmycin, though ECO-02301 differs in oxidation states in the backbone, as well as in glycosylation and the presence of the amidohydroxycyclopentenone functionality. The amidohydroxycyclopentenone moiety, postulated to be the product of intramolecular cyclization of aminolevulinic acid, is corroborated by the presence in locus 023D of an aminolevulinic acid synthase gene which presumably ensures production of the precursor aminolevulinic acid.

### EXAMPLE 6

### Identifying a Novel Polyketide From a Cryptic Biosynthetic locus via Isotope Incorporation Experiments

Streptomyces ghanaensis (NRRL B-12104) was subject to the genome scanning method described in Example 1, which resulted in the discovery in the Streptomyces ghanaensis genome of many putative natural product biosynthetic loci, seven of which were further characterized by sequence analysis and determined to be distinct biosynthetic loci. Of the seven biosynthetic loci analyzed, four contained NRPS genes and were predicted to encode the production of peptides (locus designations 009D, 009E, 009F, 009H), and two were predicted to encode for the production of a large polyketide (locus designation 009B and 009I). Based upon the genomic infomration, approximate chemical structures were predicted for the compounds encoded by loci of Streptomyces ghanaensis:

| | Mass | | | | | |
|---|---|---|---|---|---|---|
| Locus | Range | UV | Activity | Class | AA Composition | Notes |
| 009B | — | — | — | unusual | n/a | cryptic, v. small |
| | | | | polyketide | | unusual |
| 009C | >14,000 | >270 | Broad | chromoprotein | large peptide | Endyline'non- |
| | | | spectrum | enediyne | chromoprotein | covalently binds |
| | | | | | (ribosomal- | to a chromopro- |
| | | | | | tein encoded) | |
| 009D | >500 | | — | peptide | XXTXX | pentapeptide |
| 009E | >1000 | >250 | — | peptide | TFXTXXXTTX | decapeptide with |
| | | | | | | possible aromatic |
| | | | | | | moiety |
| 009F | — | — | — | peptide/ | X | cryptic, v. small |
| | | | | ketide | | |
| 009H | >1000 | 250 | — | (lipo)peptide | VFNTV*XXXX | nonapeptide, |
| | | | | | | possibly w/ N- |
| | | | | | | terminal lipid,* |
| | | | | | | N-methyl valine |
| 009I | >500 | 250 | antifungal | polyketide | n/a | 12-ketide, hygro- |
| | | | | | | lidin like, methyl- |
| | | | | | | ated, 3 conjugated |
| | | | | | | double bonds |

For instance, 009H and 009I contain gene sequences similar to genes coding for the production methylation enzymes, or methyltransferases. In the case of the hypothetical metabolites coded for by loci 009H and 009I, the sequence similarity suggested that the biosynthetic precursor for the methyl groups was S-adenosyl methionine, which is biosynthesized via methionine in primary metabolism. Partial deduction of the structures of the compounds produced by 009H and 009I suggested that they were a polypeptide and a polyketide, respectively. The proposed domain organization of the polyketide synthase of 0091 was predicted and a structure derived from this data:

[KS-AT(MM)-ACP][KS-AT(MM)-KR-ACP][KS-AT(M)-KR-ACP]

[KS-AT(MM)-ACP]

[KS-AT(MM)-KR-ACP][KS-AT(M(OCH3)M)-KR-ACP][KS-

AT(M)-DH-KR-ACP]

[KS-AT(MM)-DH-KR-ACP][KS-AT(MM)-DH-ER-KR-ACP][KS-

AT(MM)-KR-ACP]

[KS-AT(MM)-DH-KR-ACP][KS-AT(MM)-DH-KR-ACP-TE]

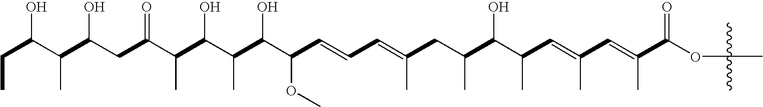

where abbreviations descibe processive enzymatic activities corresponding to ketoacyl synthase (KS), acyltransferase (AT), ketoreductase (KR), dehydratase (DH), and enoyl reductase (ER) activity, as well as acyl carrier protein (ACP) and thioesterase (TE) activity. The methoxymalonyl (mm) specificity of the sixth AT domain was discovered by domain comparison to a database of AT domains in the DECIPHER® database and supported by the presence of genesencoding enzymes known to produce methoxymalonyl-ACP, the precursor for this functionality in the metabolite encoded by locus 009I.

Thus, supplementation of multiple production media of Streptomyces ghanaensis with labeled methionine, specifically trideuteromethionine (methyl-D₃) was predicted to facilitate scanning the metabolome for the presence metabolites incorporating heavy methionine. Such metabolites incorporating heavy methionine were predicted to show mass spectral patterns consisting of a molecular ion plus a related molecular ion of lesser intensity but three daltons larger than the parent.

A metabolomics approach was subsequently used to identify conditions under which to express secondary metabolites, analyze them, and correlate them to the aforementioned biosynthetic loci based on isotopic incorporation patterns. This approach obtains analytical measurement of all low molecular weight metabolites (0-5000 Da) in a given organism at a specific time under specific culture conditions. Streptomyces ghanaensis was grown in 48 different media (AA, AB, AC, BA, CA, CB, CI, DA, DY, DZ, EA, ES, ET, FA, GA, IB, JA, KA, KE, LA, MA, MC, MU, NA, NE, NF, NG, OA, PA, PB, QB, RA, RB, RC, RM, SF, SP, TA, VA, VB, WA, WS, XA, YA, ZA), many of which are representative of media reported to support the production of a wide range of natural products. Each medium was supplemented with trideureriomethionine (methyl-D₃, 1-5 mM). Metabolites were extracted from whole cell cultures by adding of an equal volume of methanol. After removal of solid debris, the extracts were concentrated and analyzed bt the CHUMB method. Analysis of the chromatographic and bioactivity profiles indicated the presence, in a number of extracts, especially those derived from growth in medium RM, of chromatographically distinct peaks which demonstrated isotopic incorporation of trideutreromethionine as evidenced by the presence of a parent molecular ion corresponding to a mass of 574 Da plus a related ion three daltons larger than the parent ion at a ratio of parent:“+3 ion” of approximately 10:1 to 2:1.

Medium RM was selected for scale-up of fermentation to 500 mL and harvested after 10 days of growth. The general extraction protocol described elsewhere in the specification was employed and fractions 1 and 2 were found to contain the target ion. One of the methylated targets was isolated by C-18 solid phase extraction followed by C-18 HPLC. NMR data was collected for this compound including proton, carbon, COSY, HSQC, and HMBC spectra. The spectrocopic data was first used to edit the polyketide backbone derived from the locus prediction, which accelerated the elucidation of the structure. The only discrepancy between the genomic data and the NMR data was the apparent dehydration of the second hydroxyl in the predicted structure to yield the acrylate functionality. HMBC data confirmed the regiochemistry of lactone bond formation that describes the structure. Upon a search in the Dictionary of Natural Products, the isolated compound was revealed to be the known compound oxohygrolidin (shown below), which was not previously known to be produced by this organism.

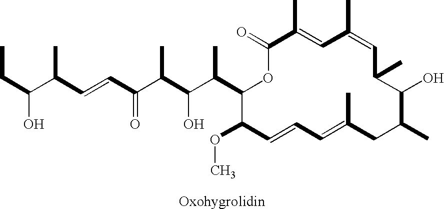

The above-described embodiments of the present invention are intended to be examples only. Alterations, modifications and variations may be effected to the particular embodiments by those of skill in the art without departing from the scope of the invention, which is defined solely by the claims appended hereto. All patents, patent applications and published references cited herein are hereby incorporated by reference in their entirety.

What is claimed is:

1. A method of identifying a secondary metabolite synthesized by a target gene cluster contained within the genome of a microorganism, which method comprises the steps of:
a) providing a microorganism containing a target gene cluster, wherein a putative or confirmed function has been attributed to at least one region of a gene in the gene cluster;
b) obtaining from the microorganism an extract containing the secondary metabolite synthesized by the target gene cluster;
c) measuring chemical, physical or biological properties of metabolites in the extract; and
d) identifying from the metabolites of step c) the secondary metabolite synthesized by the target gene cluster by comparing the chemical, physical or biological properties measured in step c) with the expected chemical, physical or biological properties of the secondary metabolite synthesized by the target gene cluster based on the putative or confirmed function attributed to the genes contained in the gene cluster.

2. The method of claim 1 wherein step b) involves growing the microorganism under multiple culture conditions to achieve expression of the target gene cluster and obtaining an extract of the fermentation broth produced under at least some of the culture conditions, and step c) involves measuring chemical, physical or biological properties of the metabolites of at least some of the extracts.

3. The method of claim 1 wherein step d) further comprises the step of comparing the chemical, physical or biological properties measured in step c) with the chemical, physical or biological properties of known compounds.

4. The method of claim 1 wherein step a) involves selecting a microorganism by reference to a knowledge repository containing information pertaining to at least one secondary metabolic gene cluster present in the genome of the microorganism.

5. The method of claim 1 wherein step b) involves growing the microorganism under multiple culture conditions selected by reference to a knowledge repository containing information pertaining to the culture conditions under which the product of at least one secondary-metabolic gene cluster is synthesized.

6. The method of claim 1 wherein the comparing of step d) is under computer control with a knowledge repository containing information pertaining to metabolites synthesized by secondary metabolic gene clusters.

7. The method of claim 3 wherein the comparing of step d) is under computer control with a knowledge repository containing information pertaining to known chemical, physical or biological properties of compounds.

8. The method of claim 1 wherein step c) involves measuring one or more properties selected from the group consisting of molecular mass, UV spectrum and bioactivity.

9. The method of claim 1 wherein the method involves the step of determining the chemical structure of the secondary metabolite.

10. The method of claim 1 wherein the method includes a step of testing the secondary metabolite produced by the target gene cluster for biological activity.

11. The method of claim 10 wherein the biological activity is antibacterial, antifungal or anticancer activity.

12. A method according to claim 1 wherein the target gene cluster is endogenous to the microorganism.

13. The method of claim 1 wherein information pertaining to any one of:
a) the association between the secondary metabolite and the target gene cluster;
b) the chemical, physical or biological properties of the secondary metabolite; and
c) the conditions under which the microorganism synthesizes the secondary metabolite;
is added to the knowledge repository.

14. A method of identifying a secondary metabolite from a pre-selected chemical family comprising the steps of:
a) establishing a correlation between the pre-selected chemical family, a structural feature of the secondary metabolite and a target gene cluster, wherein a putative or confirmed function has been attributed to at least one region of a gene in the gene cluster;
b) selecting a microorganism containing the target gene cluster;
c) obtaining from the microorganism an extract containing the secondary metabolite synthesized by the target gene cluster;
d) measuring chemical, physical or biological properties of the metabolites in the extract; and
e) identifying from the metabolites of step d) the secondary metabolite from the pre-selected chemical family by comparing the chemical, physical or biological properties of the secondary metabolite with the expected chemical, physical or biological properties based on the correlation between the pre-selected chemical family, the structural features of the secondary metabolite and the putative or confirmed function attributed to the genes contained in the gene cluster.

15. The method of claim 14 wherein step c) involves growing the microorganism under multiple culture conditions to achieve expression of the target gene cluster and obtaining an extract of the fermentation broth produced under at least some of the culture conditions, and step d) involves measuring chemical, physical or biological properties of the metabolites in at least some of the extracts.

16. The method of claim 14 wherein step e) further comprises the step of comparing the chemical, physical or biological properties measured in step d) with the chemical, physical or biological properties of known compounds.

17. The method of claim 14 wherein step a) involves reference to knowledge repository containing information pertaining to a natural product, a biological activity associated with the natural product, and a gene cluster involved in biosynthesis of the natural product.

18. The method of claim 14 wherein step b) involves selecting a microorganism by reference to a knowledge repository containing information pertaining to at least one secondary metabolic gene cluster present in the genome of the microorganism.

19. The method of claim 14 wherein step b) involves growing the microorganism under multiple culture conditions selected by reference to a knowledge repository containing information pertaining to the culture conditions under which the product of at least one secondary metabolic gene cluster is synthesized.

20. The method of claim 14 wherein the comparing of step e) is under computer control with a knowledge repository containing information pertaining to metabolites synthesized by secondary metabolic gene clusters.

21. The method of claim 16 wherein the comparing of step e) is under computer control with a knowledge repository containing information pertaining to known chemical, physical or biological properties of compounds.

22. The method of claim 14 wherein step d) involves measuring one or more properties selected from the group consisting of molecular mass, UV spectrum and bioactivity.

23. The method of claim 14 wherein the method involves the step of determining the chemical structure of the secondary metabolite.

24. The method of claim 14 wherein the method includes a step of testing the secondary metabolite produced by the target gene cluster for biological activity.

25. The method of claim 24 wherein the biological activity is antibacterial, antifungal or anticancer activity.

26. A method according to claim 14 wherein the target gene cluster is endogenous to the microorganism.

27. The method of claim 14 wherein the culture conditions under which the microorganism synthesizes the secondary metabolite synthesized by the target gene cluster are unknown.

28. The method of claim 14 wherein information pertaining to any one of:
a) the association between the secondary metabolite and the target cluster;
b) the chemical, physical or biological properties of the secondary metabolite; and
c) the conditions under which the microorganism synthesizes the secondary metabolite
is added to the knowledge repository.

29. A system for identifying a secondary metabolite synthesized by a target gene cluster contained within the genome of a microorganism, said system comprising:
a) genomic data indicating the presence of target gene cluster within a microorganism, wherein a putative or confirmed function has been attributed to at least one region of a gene in the gene cluster;
b) extraction means for obtaining an extract derived from the microorganism, said extract containing metabolites comprising the secondary metabolite synthesized by the target gene cluster;
c) an analyser for measuring chemical, physical or biological properties of metabolites in the extract; and
d) a comparator for identifying from the metabolites contained in the extract the secondary metabolite synthesized by the target gene cluster by comparing the chemical, physical or biological properties measured by the analyser with the expected chemical, physical or biological properties of the secondary metabolite synthesized by the target gene cluster based on the putative or confirmed function attributed to the genes contained in the gene cluster.

30. A system for identifying a secondary metabolite from a pre-selected chemical family, the system comprising:
a) genomic data establishing a correlation between the pre-selected chemical family, a structural feature of the secondary metabolite and a target gene cluster, wherein a putative or confirmed function has been attributed to at least one region of a gene in the gene cluster;
b) a selector for selecting a microorganism containing the target gene cluster;
c) extraction means for obtaining from the microorganism an extract containing the secondary metabolite synthesized by the target gene cluster;
d) an analyser for measuring chemical, physical or biological properties of the metabolites in the extract; and
e) a comparator for identifying from the metabolites analysed by the analyser the secondary metabolite from the pre-selected chemical family by comparing the chemical, physical or biological properties of the secondary metabolite with the expected chemical, physical or biological properties based on the correlation between the pre-selected chemical family, the structural features of the secondary metabolite and the putative or confirmed function attributed to the genes contained in the gene cluster.

31. A knowledge repository housing secondary metabolism data from a microorganism for identifying a secondary metabolite synthesized by a target gene cluster contained within the genome of the microorganism, said repository comprising:
a) genomic data confirming the presence of a target gene cluster within a microorganism, wherein a putative or confirmed function has been attributed to at least one region of a gene in the gene cluster;
b) extract-characterizing data providing chemical, physical or biological properties of metabolites contained in an extract derived from the microorganism, wherein said metabolites include a secondary metabolite attributable to the target gene cluster; and
c) comparative data representing expected chemical, physical or biological properties of the secondary metabolite synthesized by the target gene cluster, said extract-characterizing data being comparable with the comparative data for identifying from the metabolites in an extract the secondary metabolite synthesized by the target gene cluster based on the putative or confirmed function attributed to said at least one region of a gene in the gene cluster.

32. The knowledge repository of claim 31 additionally comprising culture conditions data linked to the extract-characterizing data, the culture conditions data identifying culture conditions under which a set of extract-characterizing data are obtained.

33. The knowledge repository of claim 31, wherein the comparative data comprises a known compound library holding data characterizing a chemical, physical, or biological property of a plurality of known compounds for comparison with the extract-characterizing data.

34. The knowledge repository of claim 31, wherein a prediction link is made between a record within the genomic data and a record in the comparative data when a match is established between a secondary metabolite attributable to the target gene cluster within the extract-characterizing data and the comparative data.

35. The knowledge repository of claim 31, wherein the extract-characterizing data comprises the biological property of antibacterial, antifungal or anticancer activity.

36. The knowledge repository of claim 31, additionally comprising chemical family data linked to the genomic data assigning a chemical family to genomic data indicative of a putative or confirmed function in secondary metabolic pathways leading to synthesis of a member of the chemical family.

37. A method of building a knowledge repository housing secondary metabolism data from a microorganism for identifying a secondary metabolite synthesized by a target gene cluster contained within the genome of the microorganism, said method comprising the steps of:
a) assembling genomic data confirming the presence of a target gene cluster within a microorganism, wherein a putative or confirmed function has been attributed to at least one region of a gene in the gene cluster;
b) inputting extract-characterizing data providing chemical, physical or biological properties of metabolites observed in an extract derived from the microorganism, wherein said metabolites include a secondary metabolite attributable to the target gene cluster; and
c) comparing the extract-characterizing data with comparative data representing expected chemical physical or biological properties of the secondary metabolite synthesized by the target gene cluster, so as to identify from the metabolites in an extract the secondary metabolite synthesized by the target gene cluster based on the putative or confirmed function attributed to said at least one region of a gene in the gene cluster; and
d) retaining the result of step c) by linking a secondary metabolite identified in the comparing step with the genomic data assembled in the assembling step.

38. The method of building a knowledge repository according to claim 37 wherein the step of inputting extract-characterizing data additionally comprises inputting culture conditions under which an extract is derived, and the step of retaining the result additionally comprises linking culture conditions to both the secondary metabolite identified in the comparing step and the genomic data assembled in the assembling step.

39. The method of building a knowledge repository according to claim 37 wherein the step of inputting extract-characterizing data additionally comprises inputting the biological property of antibacterial, antifungal or anticancer activity.

40. A method of building a knowledge repository housing secondary metabolism data from a microorganism for predicting secondary metabolite production from a target gene cluster based on genomic data, said method comprising:
a) assembling genomic data confirming the presence of a target gene cluster within a microorganism, wherein a putative or confirmed function has been attributed to at least one region of a gene within the gene cluster;
b) extracting a medium containing said microorganism, thereby forming an extract;
c) screening the extract for extract-characterizing data indicative of the presence or absence of a secondary metabolite attributable to the target gene cluster based on a pre-selected chemical, physical or biological property;
d) entering the extract-characterizing data into the knowledge repository;
e) comparing the extract-characterizing data with comparative data representing expected chemical, physical or biological properties of a secondary metabolite synthesized by the target gene cluster, so as to identify from the extract a secondary metabolite synthesized by the target gene cluster based on the putative or confirmed function;
f) determining the identity of a secondary metabolite extracted; and
g) affirming within the knowledge repository a correspondence between genomic data, the pre-selected chemical, physical or biological property, and the identity of the secondary metabolite, allowing a cycle of prediction of secondary metabolite production based on genomic data.

41. A memory for storing secondary metabolism data for access by an application program being executed on a data processing system for identifying a secondary metabolite synthesized by a target gene cluster contained within the genome of a microorganism, said memory comprising a data structure stored in said memory, the data structure including information resident in a database used by said application program and including (i) genomic data confirming the presence of a target gene cluster within a microorganism, wherein a putative or confirmed function has been attributed to at least one region of a gene in the gene cluster; (ii) extract-characterizing data providing chemical, physical or biological properties of metabolites contained in an extract derived from the microorganism, wherein said metabolites include a secondary metabolite attributable to the target gene cluster; and (iii) comparative data representing expected chemical, physical or biological properties of the secondary metabolite synthesized by the target gene cluster; said extract-characterizing data being comparable with the comparative data for identifying from the metabolites in an extract the secondary metabolite synthesized by the target gene cluster based on the putative or confirmed function attributed to said at least one region of a gene in a gene cluster.

42. A graphical user interface (GUI) for subscribing to a knowledge repository, said repository housing secondary metabolite data from a microorganism for identifying a secondary metabolite synthesized by a target gene cluster; said graphical user interface comprising:
a) a genomic access element for accessing from within the knowledge repository genomic data confirming the presence of a target gene cluster within a microorganism, wherein a putative or confirmed function has been attributed to at least one region of a gene in a gene cluster;
b) an extract-characterizing access element for accessing from within the knowledge repository chemical, physical or biological properties of metabolites contained in an extract derived from the microorganism, wherein said metabolites include a secondary metabolite attributable to the target gene cluster; and
c) a comparative access element for effecting a comparison of a selected chemical, physical or biological property of the secondary metabolite with chemical, physical or biological properties accessed through said extract-characterizing access element, for identifying a metabolite synthesized by the target gene cluster within a microorganism confirmed present through said genomic access element, based on the putative or confirmed function attributed to said at least one region of a gene in a gene cluster.

43. The graphical user interface according to claim 42, wherein the chemical, physical or biological properties accessed through said extract-characterizing access element comprise mass spectra, molecular mass, structural data, or biological activity characterizing a metabolite contained in an extract.

44. The graphical user interface according to claim 42 wherein said genomic access element allows searchable access to genomic data from a plurality of microorganisms.

45. The graphical user interface according to claim 42, wherein said extract-characterizing access element provides searchable access to media composition and growth conditions under which a microorganism extract was obtained.
